SEQUENCE LISTING (30 sequences)

SEQ ID NO: 1 - DNA, 21 bp, Artificial Sequence
  HNF4alpha Forward Primer
ggccaagtacatcccagcttt

SEQ ID NO: 2 - DNA, 19 bp, Artificial Sequence
  HNF4alpha Reverse Primer
cagcaccagctcgtcaagg

SEQ ID NO: 3 - DNA, 21 bp, Artificial Sequence
  Albumin Forward Primer
tttatgccccggaactccttt

SEQ ID NO: 4 - DNA, 21 bp, Artificial Sequence
  Albumin Reverse Primer
agtctctgtttggcagacgaa

SEQ ID NO: 5 - DNA, 20 bp, Artificial Sequence
  Vimentin Forward Primer
gagtccactgagtaccggag

SEQ ID NO: 6 - DNA, 20 bp, Artificial Sequence
  Vimentin Reverse Primer
acgagccatttcctccttca

SEQ ID NO: 7 - DNA, 20 bp, Artificial Sequence
  alpha-SMA Forward Primer
gtggctattccttcgttact

SEQ ID NO: 8 - DNA, 20 bp, Artificial Sequence
  alpha-SMA Reverse Primer
ggaaacgttcatttccgatg

SEQ ID NO: 9 - DNA, 20 bp, Artificial Sequence
  Col-IA1 Forward Primer
attagtaggtgtgctgtgtg

SEQ ID NO: 10 - DNA, 20 bp, Artificial Sequence
  Col-IA1 Reverse Primer
aagcgtttgcgtagtaattg

SEQ ID NO: 11 - DNA, 20 bp, Artificial Sequence
  LOX Forward Primer
gacacatcctgtgactatgg

SEQ ID NO: 12 - DNA, 20 bp, Artificial Sequence
  LOX Reverse Primer
ggggtttacactgaccttta

SEQ ID NO: 13 - DNA, 20 bp, Artificial Sequence
  TIMP1 Forward Primer
gaccaccttataccagcgtt

SEQ ID NO: 14 - DNA, 20 bp, Artificial Sequence
  TIMP1 Reverse Primer
taaacagggaaacactgtgc

SEQ ID NO: 15 - DNA, 20 bp, Artificial Sequence
  PDGFRbeta Forward Primer
cctcttggatatgccttacc

SEQ ID NO: 16 - DNA, 20 bp, Artificial Sequence
  PDGFRbeta Reverse Primer
cagctcagcaaattgtagtg

SEQ ID NO: 17 - DNA, 20 bp, Artificial Sequence
  Col-4A1 Forward Primer
attagtaggtgtgctgtgtg

SEQ ID NO: 18 - DNA, 20 bp, Artificial Sequence
  Col-4A1 Reverse Primer
aagcgtttgcgtagtaattg

SEQ ID NO: 19 - DNA, 20 bp, Artificial Sequence
  LamB1 Forward Primer
gagaggtgatatttcgtgct

SEQ ID NO: 20 - DNA, 20 bp, Artificial Sequence
  LamB1 Reverse Primer
cagaagcaatttcctcgaac

SEQ ID NO: 21 - DNA, 20 bp, Artificial Sequence
  FN Forward Primer
agtttcccattatgccgttg

SEQ ID NO: 22 - DNA, 20 bp, Artificial Sequence
  FN Reverse Primer
tcccaagacatgtgcagctc

SEQ ID NO: 23 - DNA, 20 bp, Artificial Sequence
  ITGA2 Forward Primer
ggccaggtcattatctacag

SEQ ID NO: 24 - DNA, 20 bp, Artificial Sequence
  ITGA2 Reverse Primer
ggttccaggctcatttgata

SEQ ID NO: 25 - DNA, 20 bp, Artificial Sequence
  ITGB1 Forward Primer
agagctgaagactatcccat

SEQ ID NO: 26 - DNA, 20 bp, Artificial Sequence
  ITGB1 Reverse Primer
gtgttgtgctaatgtaaggc

SEQ ID NO: 27 - DNA, 20 bp, Artificial Sequence
  CYP3A4 Forward Primer
ttttgtcctaccataagggc

SEQ ID NO: 28 - DNA, 20 bp, Artificial Sequence
  CYP3A4 Reverse Primer
cataaatcccactggaccaa

SEQ ID NO: 29 - DNA, 20 bp, Artificial Sequence
  CK-19 Forward Primer
agcatgaaagctgccttgga

SEQ ID NO: 30 - DNA, 22 bp, Artificial Sequence
  CK-19 Reverse Primer
cctgattctgccgctcactatc

(19) United States
(12) Patent Application Publication
(10) Pub. No.: US 2024/0400997 A1
(43) Pub. Date: Dec. 5, 2024

(54) TRANSFORMED HUMAN HEPATIC STELLATE CELL LINE AND USE THEREOF

(71) Applicant: KOREA RESEARCH INSTITUTE OF BIOSCIENCE AND BIOTECHNOLOGY, Daejeon (KR)

(72) Inventors: Kyung-Sook CHUNG, Daejeon (KR); Myung Jin SON, Daejeon (KR); Hyun Mi KANG, Daejeon (KR); Ho-Joon LEE, Daejeon (KR); Cho-Rok JUNG, Daejeon (KR); Ok Seon KWON, Daejeon (KR); Seonju MUN, Daejeon (KR)

(73) Assignee: KOREA RESEARCH INSTITUTE OF BIOSCIENCE AND BIOTECHNOLOGY, Daejeon (KR)

(21) Appl. No.: 18/270,078

(22) PCT Filed: Dec. 28, 2021
(86) PCT No.: PCT/KR2021/020054

§ 371 Date: Aug. 19, 2024

(30) Foreign Application Priority Data
Dec. 29, 2020 (KR) 10-2020-0186058

(51) Int. Cl. C12N 5/071; G01N 33/50

(52) U.S. Cl. CPC C12N 5/0671; C12N 5/0697; G01N 33/5067; C12N 2503/04; C12N 2513/00; G01N 2500/10

(57) ABSTRACT

The present invention relates to a transformed human hepatic stellate cell line, and a use thereof. The hepatic stellate cell line according to the present invention exhibits an improvement in drug response, and when co-cultured with hepatic stellate cells, does not exhibit a functional decrease in the hepatic cells, and thus can be advantageously used in an in vitro culture model.

### CROSS REFERENCE TO RELATED APPLICATIONS

This application is a National Stage of International Application No. PCT/KR2021/020054 filed on Dec. 28, 2021, claiming priority based on Korean Patent Application No. 10-2020-0186058 filed on Dec. 29, 2020, the entire disclosures of which are incorporated herein by reference.

### INCORPORATION BY REFERENCE OF SEQUENCE LISTING

The content of the electronically submitted sequence listing, file name: Q289151_sequence listing as filed .TXT; size: 6,151 bytes; and date of creation: Jun. 27, 2023, filed Jun. 28, 2023, is incorporated herein by reference in its entirety.

### TECHNICAL FIELD

The present invention relates to a transformed human hepatic stellate cell line and a use thereof, and in particular, to an immortalized human hepatic stellate cell line (hereinafter, referred to as HSC) which is derived from normal human liver tissue and is capable of proliferating infinitely, and a use thereof.

### BACKGROUND ART

In general, since human-derived hepatic cells have different drug-degrading ability according to race, sex, and individuals, securing primary human hepatic cells (PHH) has become an important issue in the development of a patient-specific disease model. Since the primary hepatic cells are difficult to be cultured in general 2D culture for a long period of time and the drug-degrading ability is deteriorated, 3D culture has recently attracted attention.

Meanwhile, hepatic stellate cells are known to have the next majority to hepatic cells among the cell members of liver tissue in a disease model, particularly, have a great effect on liver fibrosis and cirrhosis. There are several commercially available hepatic stellate cell lines, including LX-2, but these hepatic stellate cell lines have several limitations in that the hepatic stellate cells are difficult to be used in an actual patient-specific disease model.

First, when the hepatic stellate cells start to be cultured in vitro, they naturally differentiate into myofibroblasts, and the myofibroblasts cause liver fibrosis. In fact, it is known that the co-culture of hepatic stellate cells and hepatic cells slows the differentiation of hepatic stellate cells into myofibroblasts, but there is a limit to the regulation of differentiation into myofibroblasts.

Second, as described above, since the hepatic stellate cells have a high degree of differentiation into myofibroblasts during the in vitro culture, the reactivity to fibrosis-inducing factors deteriorates. Therefore, the hepatic stellate cells have an aspect that it is difficult to test a liver fibrosis-promoting or -inhibiting drug by preparing a liver fibrosis model. In addition, in the case of co-culturing the hepatic stellate cells with hepatic cells, when the proportion of the hepatic stellate cells increases, the activity of the hepatic cells itself decreases, thereby making it difficult to confirm a decrease in hepatocyte metabolism by a drug.

Third, the experimental model using rodents due to the difference between species in drug metabolism has a limitation, and the experimental model closest to the human body is a 3D co-culture model using primary human hepatic cells, but there is no optimized model consisting of primary human hepatic cells and hepatic stellate cells so far.

Therefore, there is a need for a hepatic stellate cell line which can induce liver fibrosis by the effect of a drug without inhibiting the function of hepatic cells in a 3D culture environment.

### DISCLOSURE OF THE INVENTION

### Technical Problem

Accordingly, the present inventors have made intensive efforts to develop a hepatic stellate cell line having a higher degree of fibrosis induction by a drug than that of a control group (conventional hepatic stellate cell line LX-2) in a drug-induced liver fibrosis experiment without inhibiting the function of hepatic cells in a normal 3D culture environment, and as a result, prepared a hepatic stellate cell line satisfying the above conditions, and confirmed that a composition including the hepatic stellate cell line could be utilized as a composition for maintaining or enhancing hepatic metabolism under normal conditions, and a composition for a drug-induced liver fibrosis model, thereby completing the present invention.

### Solution to Problem

In order to solve the above problem, one aspect of the present invention provides a transformed human hepatic stellate cell line having increased liver fibrosis activity.

Another aspect of the present invention provides an immortalized human hepatic stellate cell line derived from a human liver tissue.

Still another aspect of the present invention provides a cell composition for producing an artificial liver construct, the cell composition including the transformed human hepatic stellate cell line and hepatic cells.

One aspect of the present invention provides a liver spheroid prepared by culturing the cell composition.

Another aspect of the present invention provides an artificial liver construct produced by culturing the cell composition with a scaffold.

Yet another aspect of the present invention provides a 3D culture model including the human hepatic stellate cell line and a disease model using the 3D culture model.

Still another aspect of the present invention provides a method for preparing a liver fibrosis model, the method including 3D co-culturing of the transformed hepatic stellate cell line and hepatic cells, and a liver fibrosis model prepared by the method.

Yet still another aspect of the present invention provides a method for evaluating a liver fibrosis regulator, the method including treating the liver spheroid, the liver construct, or the liver fibrosis model with a candidate material of regulating liver fibrosis.

### Advantageous Effects of Invention

The transformed human hepatic stellate cell line according to the present invention can induce liver fibrosis by a drug without showing any inhibition of the function of hepatic cells during co-culturing with hepatic cells, and thus can be useful as a model for liver diseases such as liver fibrosis and fatty liver.

According to the present invention, the human hepatic stellate cell line can be used as single or co-cultured constituent cells in various in vitro cultures, and can also be used as a cell line for in vivo transplantation.

### BRIEF DESCRIPTION OF DRAWINGS

FIGS. 1a to 1e relate to a transformed hepatic stellate cell line prepared from human liver tissue, and immortalized human hepatic stellate cell line which can be proliferated infinitely by introducing a human telomerase reverse transcriptase (hTERT) gene and a SV40 large T-antigen gene. FIG. 1a is a microscopic image of an immortalized human hepatic stellate cell line prepared from human liver tissue; FIG. 1b shows the results obtained by identifying, with PCR, hTERT and SV40 large T-antigen expressed in the immortalized human hepatic stellate cell line; FIG. 1c shows the results obtained by identifying with immunofluorescence staining that albumin and PEPCK, which are hepatocyte markers, are weakly expressed, and hepatocyte nuclear factor 4 alpha (HNF4α) and CK18 are not expressed, and glial fibrillary acidic protein (GFAP), desmin, endoglin, and smooth muscle actin alpha (α-SMA), which are hepatic stellate cell markers, are expressed in the transformed hepatic stellate cell line; FIG. 1d shows the results obtained by confirming, with real-time PCR (RT-PCR), the expression levels of HNF4α and albumin, which are hepatocyte markers, and vimentin, which is a hepatic stellate cell marker, in the HepG2 hepatic cell line and immortalized human hepatic stellate cell line, the result showing that HNF4α and albumin, which are hepatic cell markers, are expressed low whereas vimentin, a hepatic stellate cell marker, is expressed high; and FIG. 1e shows the results obtained by confirming, with immunofluorescence staining, that GFAP, desmin, endoglin, nestin, α-SMA, type I collagen (Col-1A1), and platelet derived growth factor receptor beta (PDGFRB), which are hepatic stellate cell markers, are expressed even after subculturing an immortalized hepatic cell line.

FIGS. 2a to 2e relate to a KRIBB-HSC (K-HSC) cell line according to an embodiment of the present invention. FIG. 2a is a microscopic image of PDGFRB⁺ (CD140b⁺) cells isolated using magnetic-activated cell sorting (MACS) in order to isolate only cells having the same characteristics of hepatic stellate cells and to utilize the isolated cells as a disease model; FIG. 2b shows a result obtained by verifying, with fluorescence-activated cell sorting (FACS), that the PDGFRB⁺ cells are isolated at a purity of 98.3%; FIG. 2c shows a result obtained by identifying insertion sites and variants of the HSC/PDGFRB⁺ cells through whole genome sequencing; FIG. 2d shows a result obtained by confirming, with immunofluorescence staining, the expression levels of GFAP, α-SMA, desmin and PDGFRB, which are hepatic stellate cell markers, in the K-HSC cell line (PDGFRB⁺ cells); and FIG. 2e is a microscopic image of GFP-K-HSC prepared using a retrovirus including a green fluorescent protein (GFP) gene for convenience in future utilization.

FIGS. 3a to 3d show comparison between the immortalized human hepatic stellate cell line and LX-2, a conventional hepatic stellate cell line. FIG. 3a shows the results obtained by measuring and comparing the expression levels of hepatic fibroblast-specific markers with RT-PCR after subculturing 5 times each of the immortalized human hepatic stellate cell line and LX-2 in Dulbecco's Modified Eagle Medium (DMEM) and Williams E medium, respectively;

FIG. 3b shows the results obtained by confirming, with immunofluorescence staining, the expression of α-SMA, Col-IA1, PDGFR-β, which are hepatic stellate cell markers, and collagen type IV alpha 3 (Col-4A3), which is a extracellular matrix marker; FIG. 3c shows the results obtained by analyzing differences in the expression of collagen-type IV alpha 1 (Col-4A1), laminin subunit beta 1 (LamB1), fibronectin (FN), integrin subunit alpha 2 (ITGA2), and integrin subunit beta 1 (ITGB1), which are major proteins of extracellular matrix, for each cell and each medium; and FIG. 3d shows the results obtained by analyzing, with RT-PCR, the expression levels of HGF, which is a hepatocyte growth factor, and TGF-β1, which is a liver fibrosis-inducing factor, in HepaRG, which is an undifferentiated hepatic cell line cultured in Williams E medium, the immortalized human hepatic stellate cell line, and LX-2, for each cell, the results showing that the expression levels of HGF and TGF-β1 in the immortalized human hepatic stellate cell line and LX-2 are lower than those in undifferentiated HepaRG, but LX-2 showed higher expression level of TGF-β1 than the immortalized human hepatic stellate cell line, indicating that the degree of differentiation into myofibroblasts is higher.

FIGS. 4a and 4b relate to a liver spheroid prepared by co-culturing the immortalized hepatic stellate cell line or LX-2, which is a conventional hepatic stellate cell line, together with a hepatic cell line. FIG. 4a shows the results obtained by confirming, with RT-PCR, the expression levels of HNF4α, albumin, cytochrome 3A4 enzyme, which are markers for the differentiation into hepatic cells, and CK-19 which is a marker for the differentiation into cholangiocytes in a spheroid (HSCs Co) prepared by culturing the hepatic cell line HepaRG and the immortalized hepatic cell line at a ratio of 4:6 and 8:2, and a spheroid (LX-2 Co) prepared by culturing the hepatic cell line HepaRG and LX-2 at a ratio of 4:6 and 8:2, and the spheroid prepared by culturing the hepatic cell line HepaRG and the immortalized hepatic cell line at a ratio of 4:6 and 8:2 showed that the expression levels of HNF4α, albumin, and cytochrome 3A4 enzyme, which are markers for the differentiation into hepatic cells, are improved (from a ratio of 4:6) or similar to the expression level (at a ratio of 8:2), and thus does not inhibit the differentiation of the hepatic cell as compared to the spheroid prepared by co-culturing LX-2 and the hepatic cell line (HepaRG), and shows that the expression levels of HNF4α, albumin, and CYP3A4 enzyme, which are markers for the differentiation into hepatic cells, and CK-19, which is a marker for the differentiation into cholangiocytes, are all high, and thus does not exhibit a functional decrease in the hepatic cells, which is superior to LX-2; and FIG. 4b shows the results obtained by confirming, with RT-PCR, the degree of change of the fibrosis marker of the hepatic stellate cells in the produced spheroid, and the spheroid prepared by co-culturing LX-2 and the hepatic cell line (HepaRG) showed that the marker for the differentiation into hepatic cells was generally expressed lower than the spheroid prepared by culturing the hepatic cell line HepaRG and the immortalized hepatic stellate cell line at a ratio of 4:6 and 8:2, and the degree of initial liver fibrosis is similar to that of LX-2 unlike the 2D culture. The degree of expression was compared by being quantified according to the cell ratio.

FIGS. 5a to 5e relate to whether a myofibroblast marker is activated during 2D culture of the immortalized human hepatic stellate cell line or LX-2 and the hepatic cell line, and to liver analogs prepared by co-culturing the immortalized hepatic stellate cell line or LX-2 and the hepatic cell line (HepaRG) in fibrin gel. FIG. 5a shows the results obtained by confirming, with immunochemical staining, the expression of PDGFRβ, which is a hepatic stellate cell marker, and Collagen, which is a myofibroblast marker, after the 2D culture of the immortalized human hepatic stellate cell line or LX-2; FIG. 5b shows the results obtained by confirming that the immortalized human hepatic stellate cell line has poor cell adhesion ability to a hybrid hydrogel compared to LX-2 when the hepatic cell line HepaRG, LX-2, and the immortalized human hepatic stellate cell line are encapsulated at a ratio of 1:1 in various concentrations of fibrin gels and the hybrid hydrogel formed of polyethylene glycol, which is an artificial material/hyaluronic acid/RGD (Arg-Gly-Asp) peptide, and 3D co-cultured for 21 days, and then the network formation is observed through a confocal microscope by means of actin-phalloidin fluorescence staining; FIG. 5c shows the results obtained by measuring the activity of liver analogs prepared by co-culturing the immortalized hepatic stellate cell line or LX-2 and the hepatic cell line (HepaRG) in various concentrations of fibrin gels, and normalizing the measurements with ATP, the results showing that the fibrin gel affinities of the cells constituting the liver analogs prepared by co-culturing the immortalized hepatic stellate cell line or LX-2 and the hepatic cell line (HepaRG) in fibrin gels are similar; and FIGS. 5d and 5e show the results obtained by measuring the amount of albumin secreted and the activity of cytochrome p450 3A4 (CYP3A4) enzyme induced with rifampicin in a liver analog prepared by culturing hepatic cell line (HepaRG) in fibrin gel, a liver analog prepared by co-culturing the hepatic cell line (HepaRG) and the immortalized hepatic stellate cell line in fibrin gel, and a liver analog prepared by co-culturing LX-2 and hepatic cell line (HepaRG) in fibrin gel, and normalizing the measurements with ATP.

FIGS. 6a to 5d relate to liver analogs prepared by 3D co-culturing the immortalized human hepatic stellate cell line or LX-2 together with a hepatic cell line in laminin-coated biosilk, which is a natural material. FIG. 6a shows the results obtained by encapsulating/attaching the hepatic cell line HepaRG and the immortalized human hepatic stellate cell line to laminin-coated biosilk at a ratio of 1:1; FIG. 6b shows the results obtained by measuring the activity of the respective cells in the case of culturing the hepatic cell line HepaRG in the laminin-coated biosilk, the case of co-culturing the hepatic cell line HepaRG and immortalized human hepatic stellate cell line in the laminin-coated biosilk, and the case of co-culturing the hepatic cell line HepaRG and LX-2 in the laminin-coated biosilk, and normalizing the measurements with ATP, the results showing that the activity of the cells was similar even though the hepatic cell line HepaRG and the immortalized human hepatic stellate cell line were co-cultured in the laminin-coated biosilk or the hepatic cell line HepaRG and LX-2 were co-cultured in the laminin-coated biosilk; and FIGS. 6c and 6d show the results obtained by measuring the activity of albumin and CYP3A4 enzyme each after the culture of HepaRG for 5 days, the co-culture of HepaRG and LX-2 for 5 days, or the co-culture of HepaRG and the immortalized human hepatic stellate cell line for 5 days, and normalizing the measurements with ATP, the results showing that the experimental group including the immortalized human hepatic stellate cell line increases hepatic cell activity in the co-culture environment compared to the experimental group including LX-2.

FIGS. 7a to 7d show the results obtained by co-culturing the immortalized human hepatic stellate cell line or LX-2 with the hepatic cell line HepaRG at a ratio of 6:4 in hybrid hydrogels having various macromer concentrations and initial stiffness, and then quantifying the degree of liver fibrosis progress with RT-PCR, wherein the groups are composed of a control group (cont) to which a drug is not administered, an experimental group to which one of two drugs, acetaminophen (apap) and methotrexate (MTX), is administered, and a positive control group to which TGF-β1 inducing fibrosis is administered. The degree of fibrosis progress of the hepatic stellate cell lines was confirmed through the expression levels of smooth muscle actin alpha (α-SMA) (FIG. 7a), type I collagen (Col-1A1) (FIG. 7b), lysyl oxydase (LOX) (FIG. 7c), and tissue inhibitor of matrix metalloproteinase 1 (TIMP1) (FIG. 7d) of matrix metalloproteinase.

FIGS. 8a to 8d are graphs for comparing changes in the differentiation (HNF4u) of co-cultured hepatic cell line HepaRG (FIG. 8a), changes in albumin production capacity (FIG. 8b), and differences in the expression of fibronectin, which is a cell adhesion protein, when the immortalized human hepatic stellate cell lines and LX-2's were each co-cultured with the hepatic cell line in hybrid hydrogels having various macromer concentrations and initial stiffness, respectively; and FIG. 8d shows the results obtained by confirming the shape difference in the hydrogel during a single 3D culture of the immortalized human hepatic stellate cell line and LX-2 for 24 days without drug treatment, the results showing that the immortalized human hepatic stellate cell line exhibits condensation of cells in long-term culture compared to LX-2.

FIGS. 9a to 9d show the results obtained by quantifying the expression of a marker for liver fibrosis according to whether hepatocyte growth factor (HGF) is administered after the immortalized human hepatic stellate cell lines and LX-2's were each encapsulated together with the hepatic cell line HepaRG at a ratio of 1:1 in hydrogels having two macromer concentrations simulating normal liver tissue stiffness (4.5% macromer) or liver cirrhosis tissue stiffness (8% macromer) and co-cultured. As a control group, the expression of a marker in a 2D monolayer before hydrogel encapsulation was compared. The expression level of the fibrosis marker in the hepatic stellate cell lines shows the results confirming that the expression of type I collagen (Col-1A1) (FIG. 9a), smooth muscle actin alpha (α-SMA) (FIG. 9b), lysyl oxidase (LOX) (FIG. 9c), and tissue inhibitor of matrix metalloproteinase 1 (TIMP1) (FIG. 9d) have statistically significant changes according to the macromer concentration and whether to administer the hepatocyte growth factor HGF.

FIGS. 10a to 10c show the results obtained by comparing the degree of liver injury simulation according to 3D cultures in which the immortalized human hepatic stellate cell lines and LX-2's were encapsulated or self-associated with the hepatic cell lines (HepaRG) at a ratio of 1:1, and applied to the various 3D cultures (cell self-association spheroids, hydrogels, and biodegradable polylactide nanoparticle-encapsulating hydrogels (NP-hydrogels)), and then methotrexate (MTX) was administered for liver injury simulation, and TGF-β1 was administered for liver fibrosis induction. The degree of liver simulation of the immortalized human hepatic stellate cell line and LX-2 was confirmed by the activity of the CYP3A4 enzyme (FIG. 10a), the sensitivity of drug metabolism (fold change, the CYP3A4 enzyme activity ratio of the 3D cultures with rifampicin drug administration and the 3D cultures exposed to simple DMSO) (FIG. 10b), and the albumin production capacity (FIG. 10c), and the LX-2 and the immortalized human hepatic stellate cell line were found to have low CYP3A4 enzyme activity and albumin production capacity in the form of a spheroid, but were found that the CYP3A4 enzyme activity and functional decrease were simulated by MTX and TGF-β1 in the hydrogels and the NP-hydrogels including biodegradable polylactide nanoparticles.

FIGS. 11a to 11f show the results obtained by confirming affinities, hepatocellular functions, and liver injury simulation of various adhesion proteins of immortalized human hepatic stellate cell lines in a 3D hydrogel environment. FIG. 11a shows the results obtained by observing, through an optical fluorescence microscope, the cell lines after applying the immortalized human hepatic stellate cell lines and LX-2's to hydrogels encapsulating RGD, fibronectin, or laminin, which are adhesion proteins, followed by the 2D culture for 12 hours; FIG. 11b shows results obtained by observing, through a confocal microscope, the cell lines after 3D culturing of the immortalized human hepatic stellate cell lines or LX-2's together with the hepatic cell line HepaRG in hydrogels encapsulating RGD, fibronectin, or laminin for 5 days; and FIGS. 11c to 11f show the results obtained by confirming the activity of CYP3A4 enzyme (FIG. 11c), albumin production capability quantified by RT-PCR (FIG. 11d), and the expression levels of smooth muscle actin alpha (α-SMA) (FIG. 11e) and type I collagen (Col-1A1) (FIG. 11f) after the 3D culture for 7 days of the immortalized human hepatic stellate cell lines and LX-2's with the hepatic cell line HepaRG at a ratio of 1:1 in hydrogels encapsulating RGD, fibronectin, or laminin and the administration of TGF-1.

FIGS. 12a to 12c show the results obtained by confirming the changes in appearance in the hydrogels (FIG. 12a), the albumin production capacity (FIG. 12b) on day 2 of culture, day 11 of culture, and day 24 of culture, and the difference in the expression of the liver fibrosis markers (FIG. 12c) after the 24-day culture by encapsulating the immortalized human hepatic stellate cell lines and the primary human hepatic cells in hydrogels or hydrogels (NP-hydrogels) including biodegradable polylactide nanoparticles at a ratio of 1:1, and adding TGF-β1, which is a fibrosis-inducing factor, the results showing that the hydrogel system is more suitable for liver simulation using the primary human hepatic cells and the immortalized human hepatic stellate cell lines.

FIGS. 13a and 13b show the results obtained by confirming the expression of the fibrosis marker (FIG. 13a) of the hepatic stellate cells of the spheroids according to the presence or absence of exposure of TGF-β1 and the differentiation degree of the hepatic cells (FIG. 13b) of the spheroids after setting, as one cycle, that the immortalized human hepatic stellate cell lines or LX-2's were self-associated with the differentiated hepatic cell line HepaRG at a ratio of 6:4 (hepatic cells:hepatic stellate cells) in the presence of physical stimulus through the rotary shaking (70 rpm) in Williams E medium containing 2% serum for 3 days to form spheroids in the form of 3D culture, and then converted to serum-free Williams E medium, and cultured for 4 days, then TGF-β1, a fibrosis-inducing factor, was added to the resulting spheroids, and the spheroids were exposed to the TGF-β1 for 24 hours, and then exposed to the growth factor-free medium for 24 hours, and repeating five cycles.

FIGS. 14a and 14b show the results obtained by confirming the cell network formation (FIG. 14a) and the expression (FIG. 14b) of the hepatic stellate cell marker and the liver fibrosis marker after the 18-day culture of groups by encapsulating the immortalized human hepatic stellate cell lines or LX-2's with the differentiated hepatic cell line HepaRG at a ratio of 1:1 in 4% hydrogels, and dividing the groups into experimental groups to which TGF-β1, a fibrosis-inducing factor, and inhibitor A thereof are administered, respectively or simultaneously, and a control group to which they are not administered.

### BEST MODE FOR CARRYING OUT THE INVENTION

Hereinafter, the present invention will be described in more detail.

One aspect of the present invention provides a transformed human hepatic stellate cell line into which a human telomerase reverse transcriptase (hTERT) gene and a SV40 large T-antigen gene, or a gene encoding a polypeptide of the hTERT or SV40 large T-antigen are introduced and which shows positive for the expression of a platelet-derived growth factor receptor beta (PDGFR-0) marker.

Meanwhile, the information on the gene sequences of the hTERT and SV40 large T-antigen of the present invention or the polypeptide amino acid sequence of the hTERT or SV40 large T-antigen may be obtained from a known database (e.g., GenBank of NCBI). In addition, the nucleotide sequences of the hTERT and SV40 large T-antigen or the polypeptide amino acid sequence of the hTERT or SV40 large T-antigen may have at least 70%, at least 75%, at least 80%, at least 85%, at least 90%, at least 95% or at least 99% sequence identity to the nucleotide sequence or amino acid sequence information which may be obtained from the known database.

In addition, the transformed human hepatic stellate cell line may be an immortalized human hepatic stellate cell line capable of proliferating indefinitely. According to an embodiment of the present invention, it was confirmed that the transformed human hepatic stellate cell line can be subcultured at least 19 times, at least 31 times, or up to 62 times.

The present inventors have found that the hTERT gene and the SV40 large T-antigen gene are introduced to the hepatic stellate cells isolated from a human, and thus the human hepatic stellate cell lines are transformed, and the transformed human hepatic stellate cell lines have an increase in liver fibrosis activity, and does not inhibit the activity of hepatic cells when co-cultured with the hepatic cells, and thus have completed the present invention.

In the present invention, the introduction of the hTERT gene and the SV40 large T-antigen gene may be performed by any method known in the art. Specifically, the transformation may be performed with an expression vector including a nucleotide encoding hTERT and a nucleotide encoding a SV40 large T-antigen. The nucleotides may be delivered to a single expression vector or to different expression vectors, respectively. The expression vector may be a viral vector, for example, a lentiviral vector.

In the present invention, PDGFR-0 is one of the receptors that bind to PDGF, which is the most potent division and proliferation-promoting cytokine of the hepatic stellate cells, and may be a marker showing liver fibrosis activity of the hepatic stellate cells.

The transformed human hepatic stellate cell line may include at least 80%, at least 90%, at least 95%, or at least 98% of cells that are positive for the PDGFR-0 marker. According to an embodiment of the present invention, the transformed human hepatic stellate cell line has a purity of about 98.3% of PDGFRB⁺ cells.

The transformed human hepatic stellate cell line may have overexpressed smooth muscle actin alpha (α-SMA).

The transformed human hepatic stellate cell line may exhibit increased expression of a hepatic stellate cell marker. Specifically, the expression of one or more hepatic stellate cell markers selected from the group consisting of α-SMA, lysyl oxidase (LOX), tissue inhibitor of matrix metalloproteinase 1 (TIMP1), and PDGFR-0 may be increased.

In addition, the transformed human hepatic stellate cell line may exhibit increased expression of an extracellular matrix marker. Specifically, the expression of one or more extracellular matrix markers selected from the group consisting of collagen type IV alpha 1 (Col-4A1), collagen type IV alpha 3 (Col-4A3), laminin subunit beta 1 (LamB1), fibronectin (FN), integrin subunit alpha 2 (ITGA2), and integrin subunit beta 1 (ITGB1) may be increased.

According to an embodiment of the present invention, the transformed human hepatic stellate cell line may have the expression of the hepatic stellate cell and/or extracellular matrix marker of at least 1.5 times, at least 2 times, at least 3 times, at least 4 times, at least 5 times, or at least 10 times, compared to an LX-2 cell line, which is a known human hepatic stellate cell line. In addition, the expression of the hepatic stellate cells and/or the extracellular matrix marker may be up to 25 times compared to the LX-2 cell line, which is a known human hepatic stellate cell line. In particular, the transformed human hepatic stellate cell line may have an increased expression of α-SMA by 10 times or more, as compared to the LX-2 cell line. In this case, the cell line may be cultured in a DMEM medium or a Williams E medium.

The transformed human hepatic stellate cell line may exhibit liver fibrosis activity. The expression “exhibiting liver fibrosis activity” as used herein means that the hepatic stellate cells induce liver fibrosis, such as cell proliferation, contraction, inflammatory cytokine secretion (chemoattraction), migration, and secretion of extracellular matrix.

Another aspect of the present invention provides a cell composition for producing an artificial liver construct, the cell composition including the transformed human hepatic stellate cell line and hepatic cells.

The term “artificial liver construct” as used herein refers to a 3D cell mass that simulates human liver functions. The artificial liver construct may be a liver spheroid formed by direct binding of hepatic stellate cells and hepatic cells. In addition, the artificial liver construct may be a 3D liver analog formed by 3D culture of hepatic stellate cells and hepatic cells with a scaffold (support).

The transformed human hepatic stellate cell line is the same as described above.

The hepatic cells may be primary human hepatic cells, immortalized hepatic cell lines, or stem cell-derived hepatic cells, but are not limited thereto.

The cell composition may further include a medium for maintaining or culturing the hepatic stellate cell lines and hepatic cells.

Yet another aspect of the present invention provides a liver spheroid prepared by culturing the cell composition.

The liver spheroid may simulate a biological environment in a liver disease model such as liver fibrosis, liver disease associated with liver fibrosis, or fatty liver. Therefore, the liver spheroid may be utilized for drug evaluation such as efficacy, safety, and toxicity of candidate drugs in a liver disease model such as liver fibrosis, liver disease associated therewith, or fatty liver.

According to an embodiment of the present invention, even when the transformed human hepatic stellate cell line was in direct contact with the hepatic cells during the culturing of the cell composition, the liver spheroid exhibited increased or similar expression of HNF4α, albumin, and CYP3A4 enzyme, which are markers for the differentiation into hepatic cells, compared to those of the culture of hepatic cells alone, and high expression of all of HNF4α, albumin and CYP3A4 enzyme, which are markers for the differentiation into hepatic cells, and CK-19, which is a marker for the differentiation into cholangiocytes, compared to the spheroid co-cultured with the LX-2, and thus the liver spheroid was confirmed to exhibit excellent hepatocellular function. In addition, the liver spheroid exhibited initial liver fibrosis activity because the expression levels of Col-1A1, α-SMA, TIMP1, and LOX, which are markers for the fibrosis progress of the hepatic stellate cells, are similar to those of the spheroid co-cultured with the LX-2.

Still another aspect of the present invention provides an artificial liver construct produced by culturing the cell composition with a scaffold.

The scaffold may be a 3D matrix based on natural materials or artificial materials. Specifically, the scaffold may be a polyethylene glycol-containing hydrogel, a fibrin gel, or biosilk, but is not limited thereto.

The scaffold-based culture may be performed by crosslinking or coating an RGD (Arg-Gly-Asp) peptide, fibrin, or laminin in the scaffold.

The artificial liver construct may simulate a biological environment in a liver disease model such as liver fibrosis, liver disease associated with liver fibrosis, or fatty liver. Thus, the artificial liver construct may be utilized in drug evaluation such as efficacy, safety, and toxicity of candidate drugs in a liver disease model, such as liver fibrosis, liver disease associated with liver fibrosis, or fatty liver.

According to an embodiment of the present invention, the artificial liver construct is confirmed to exhibit high albumin secretion and CYP3A4 enzyme activity in the hepatic cells as compared to the artificial liver construct obtained by co-culturing with the LX-2 cell line, thereby exhibiting excellent drug metabolizing ability and hepatic cell activity.

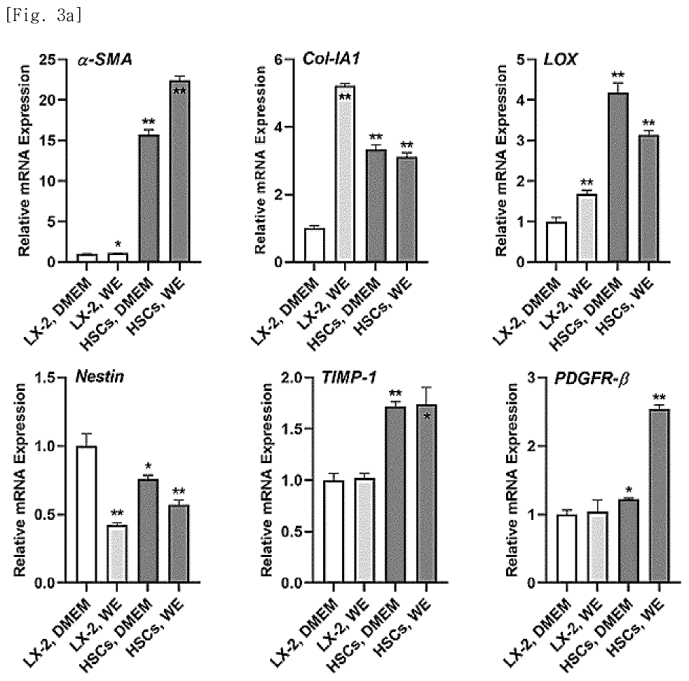

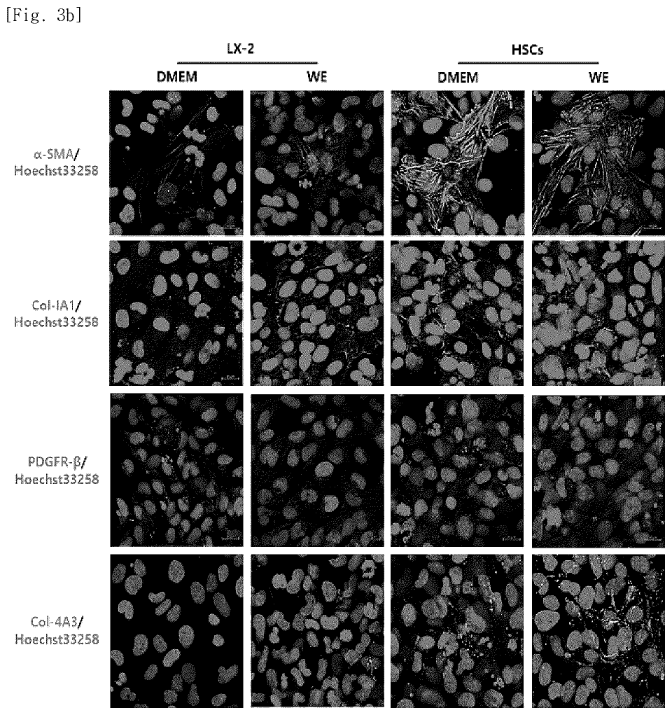

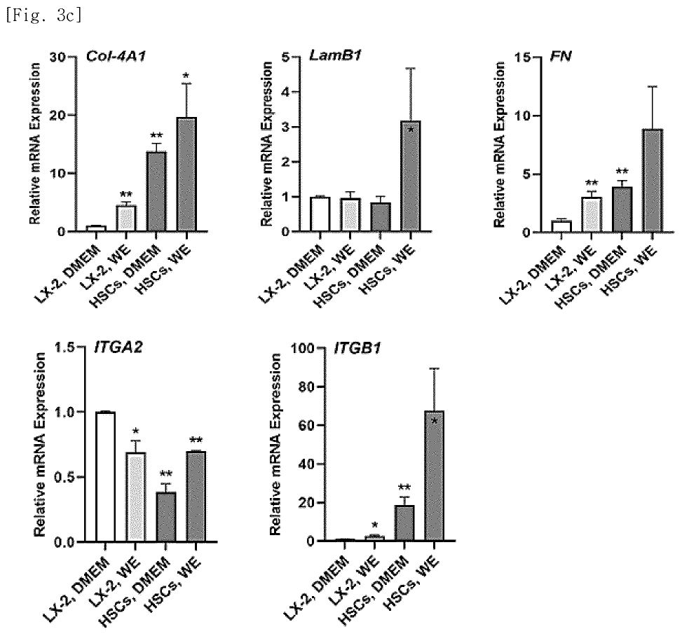

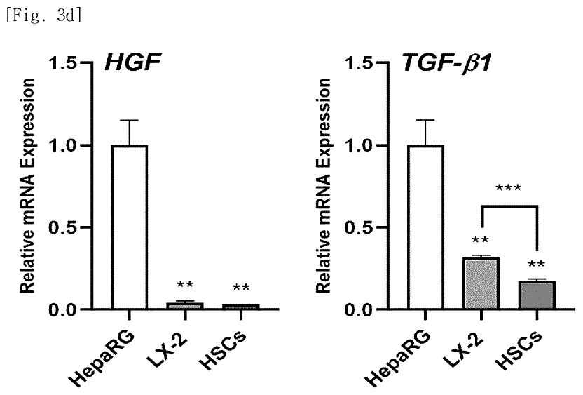

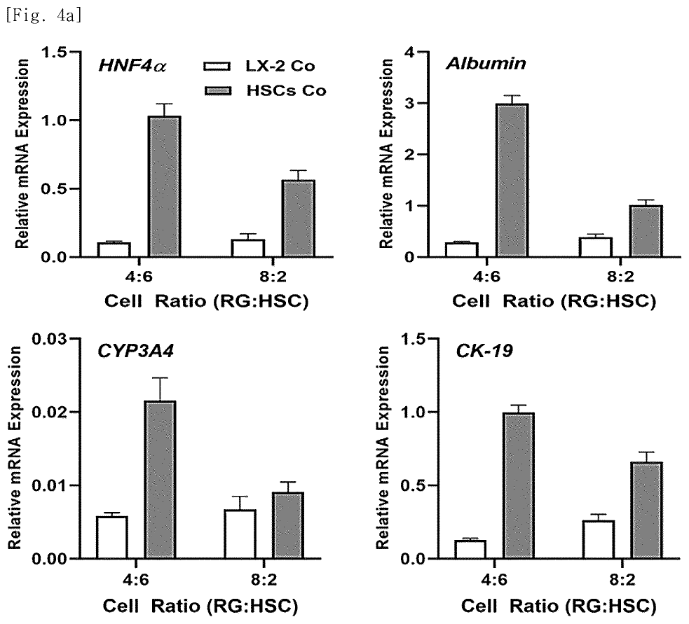

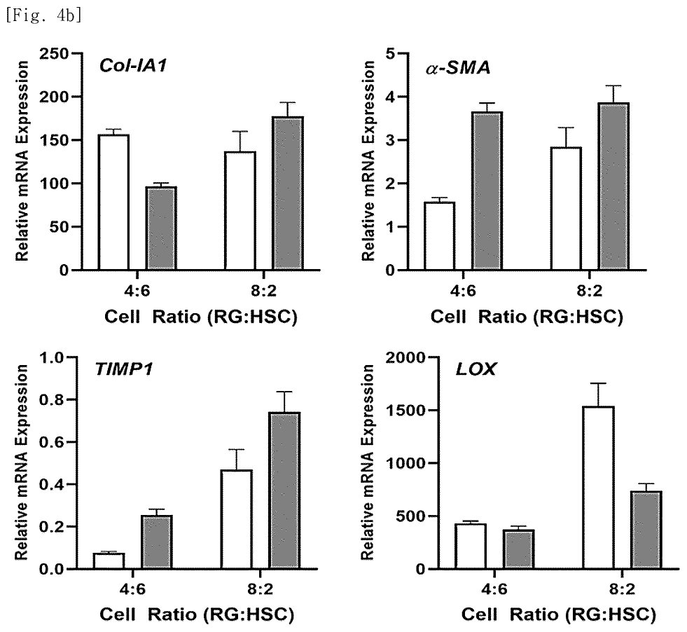

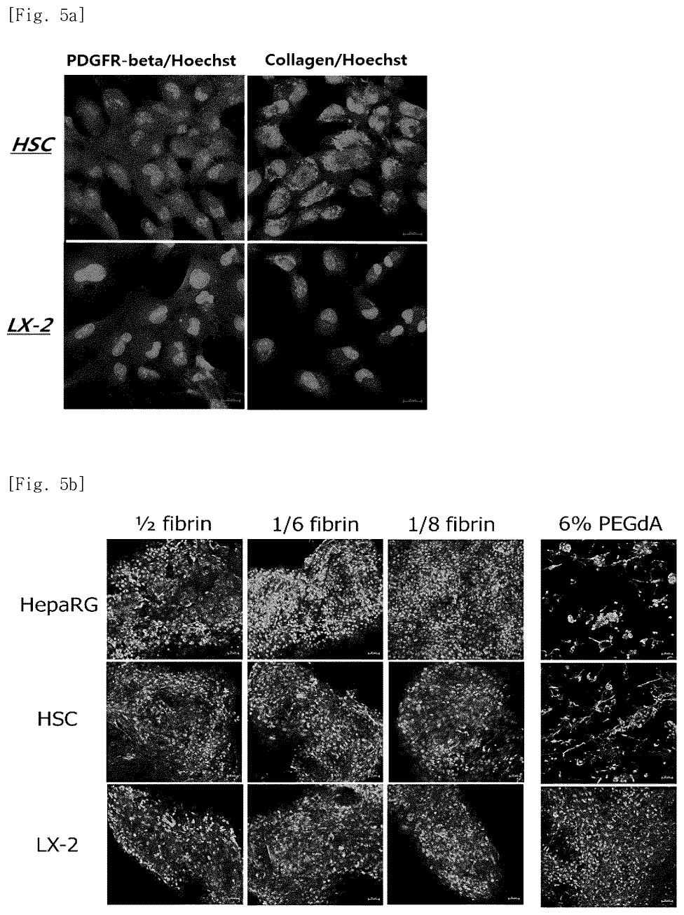

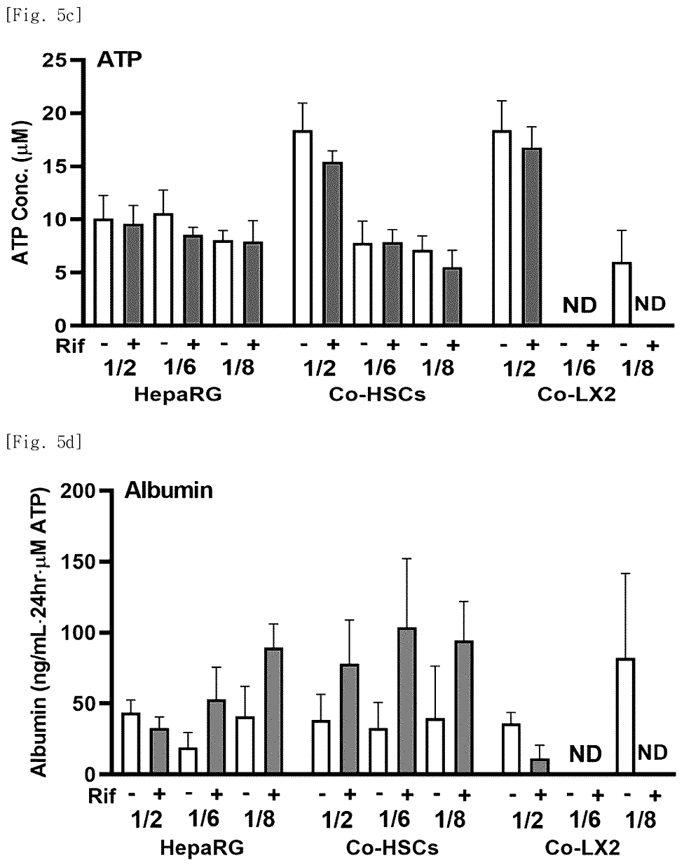

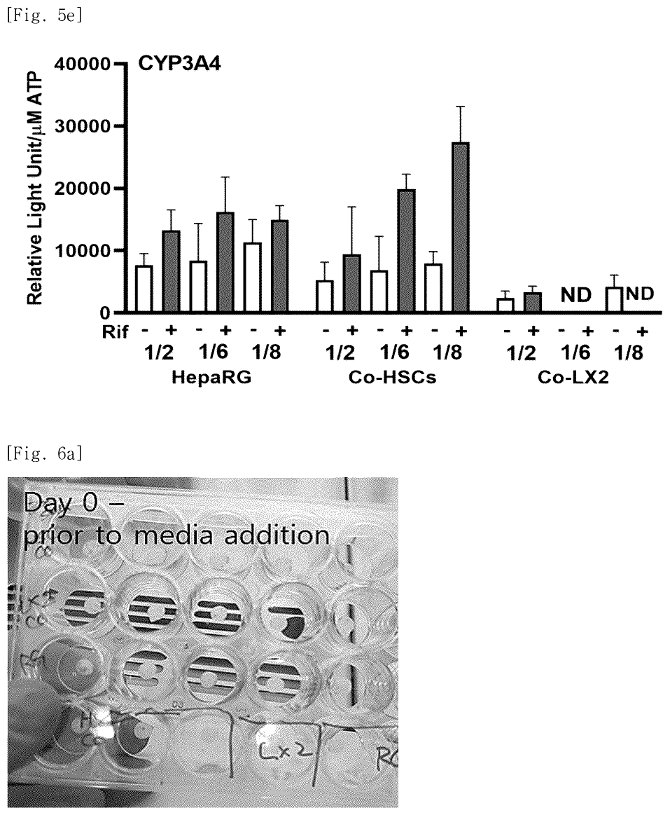

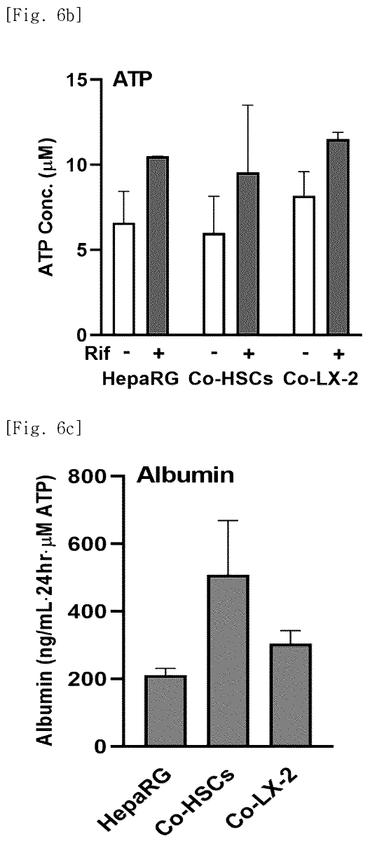

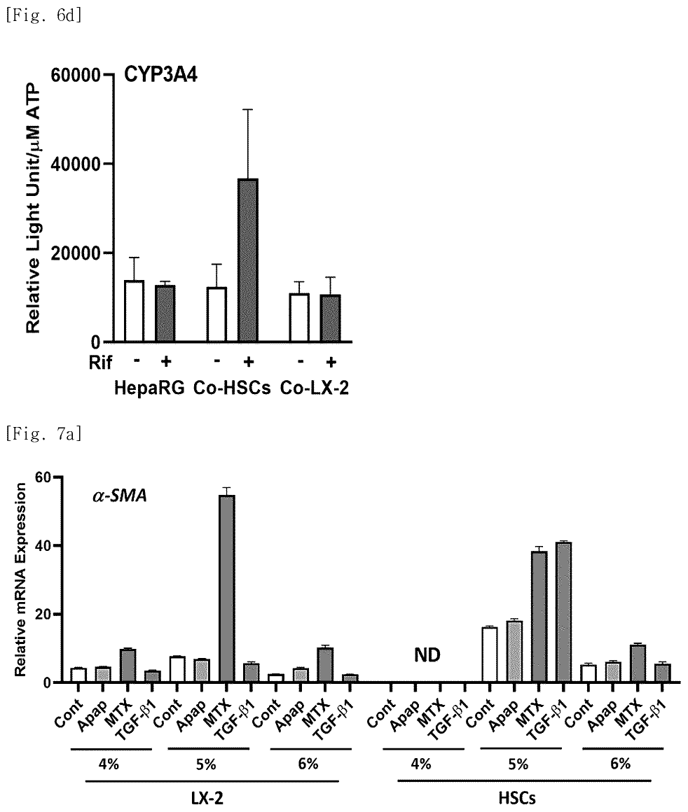

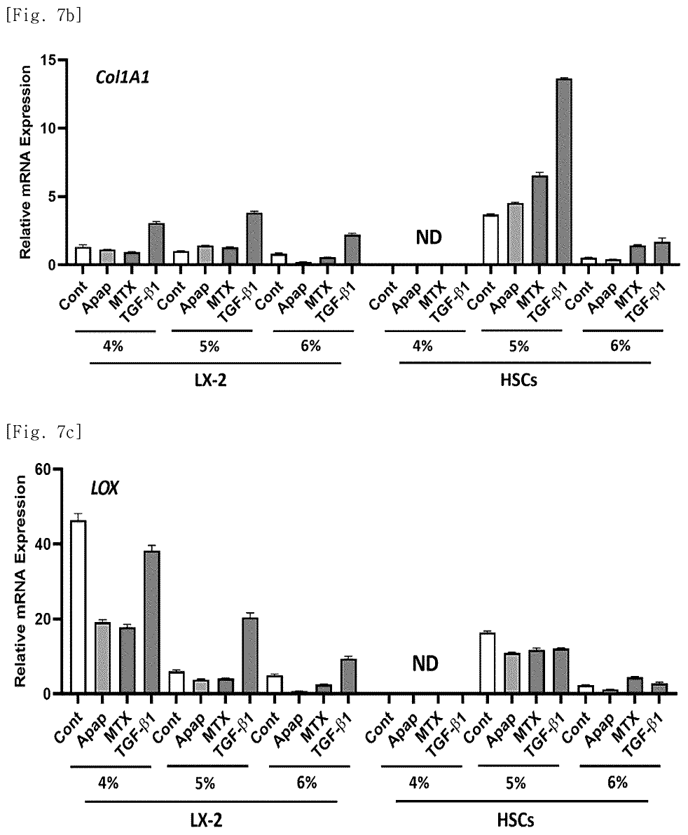

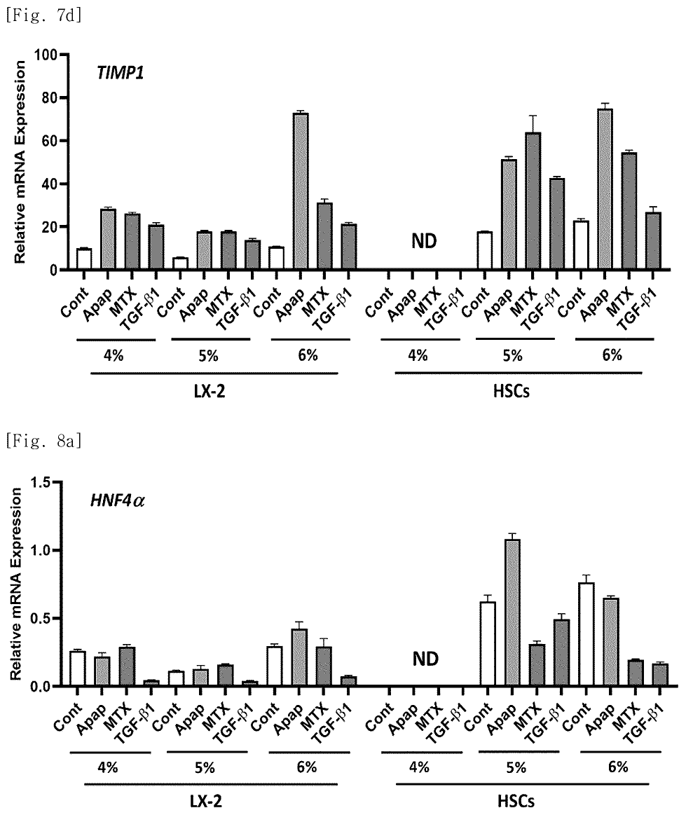

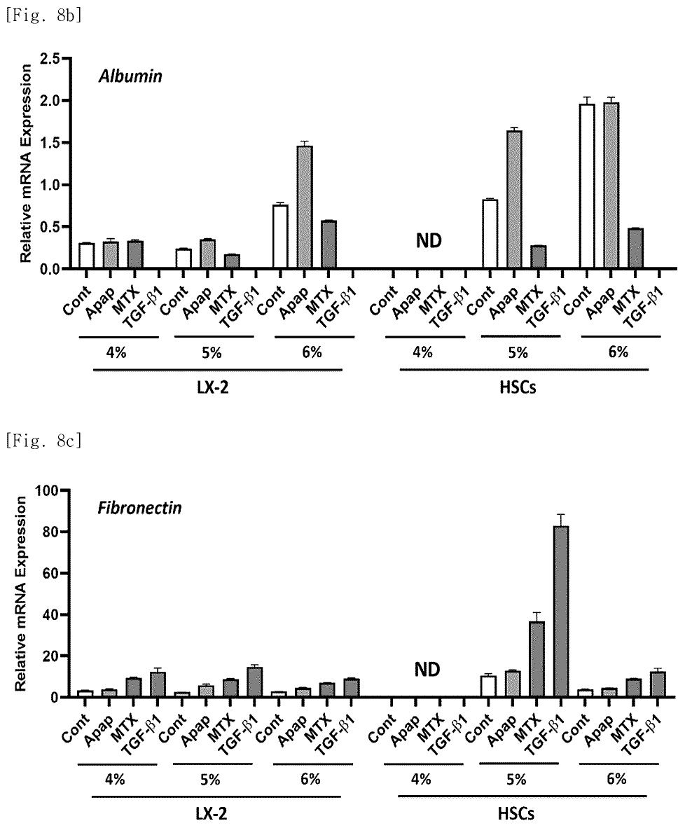

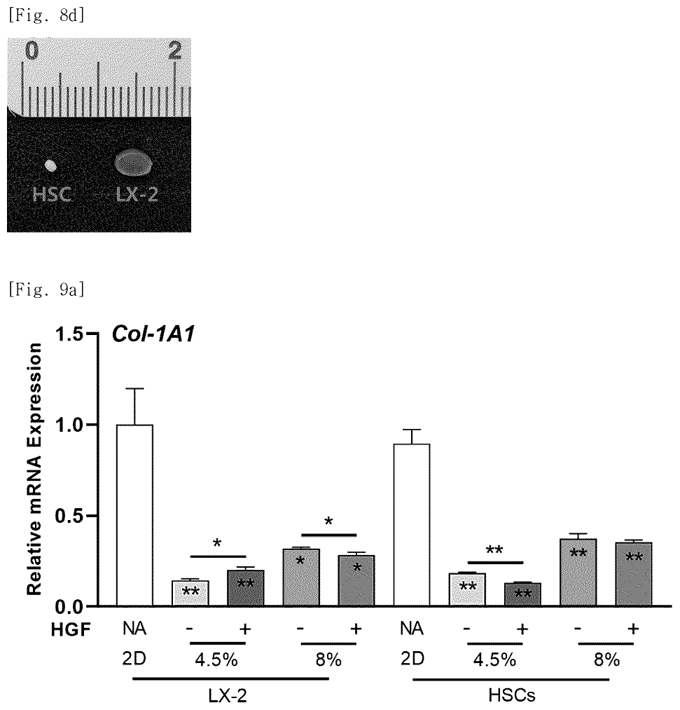

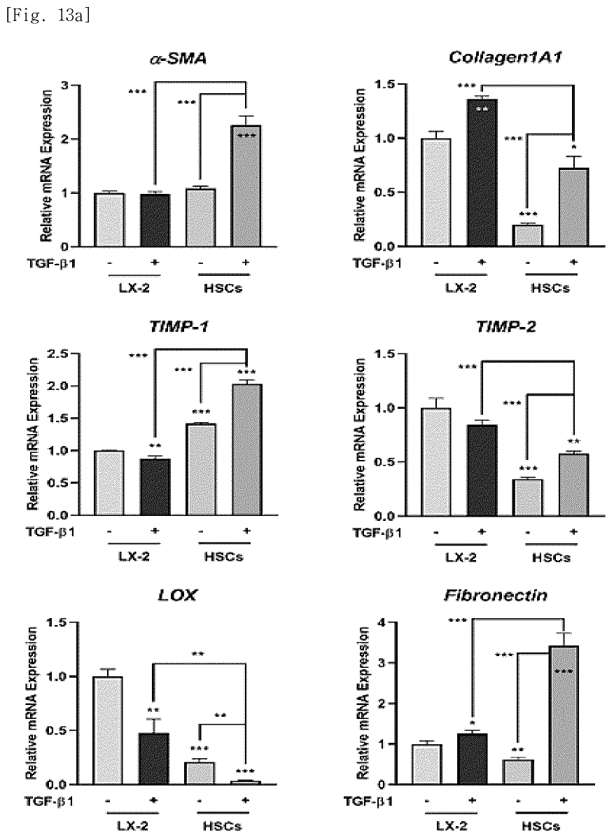

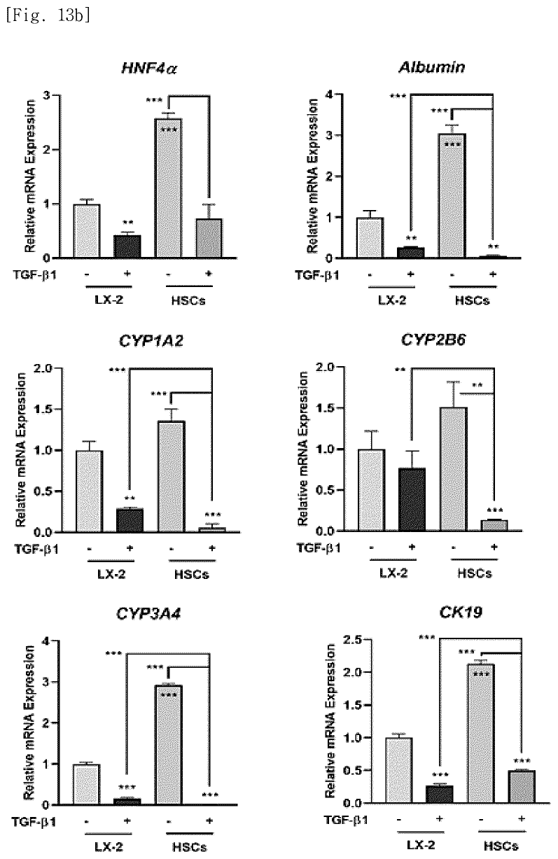

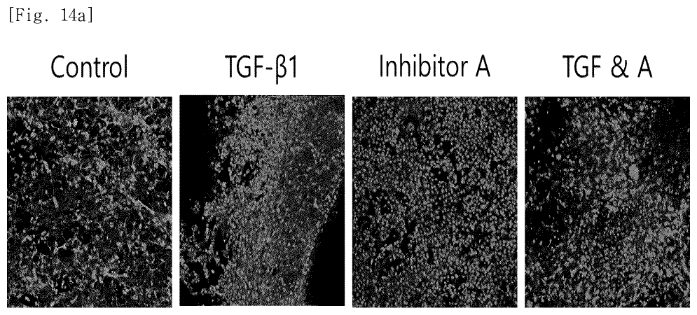

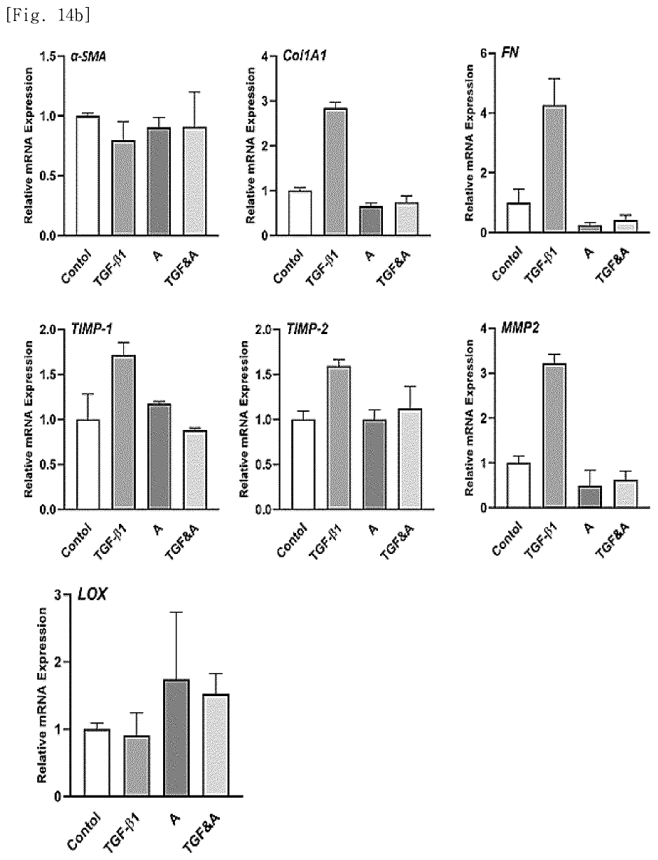

When compared with the LX-2 cell line, which is a conventional hepatic stellate cell line, it was confirmed that α-SMA involved in tissue contraction was overexpressed (FIGS. 3a and 3b) in the hepatic stellate cell line of the present invention, and it may be seen from this that unlike the LX-2 cell line, stiffness simulation of the disease model is possible through the contraction of the 3D hydrogel cultures (FIGS. 7a to 7d and 8d). Meanwhile, it was confirmed that the expression of type IV collagen and laminin (LamB1), which play important roles in tissue regeneration for recovering from a disease or injury, in the hepatic stellate cell line of the present invention was significantly increased compared to the LX-2 cell line, which is the existing cell line (FIG. 3c), and this may be a cause of high expression and activity maintenance of a marker for the differentiation into the hepatic cell line compared to the LX-2 cell line (FIG. 4; FIGS. 5d and 5e; FIGS. 6c and 6d; and FIG. 13b). These characteristics indicate that the hepatic stellate cell line of the present invention may be applied to the study of liver disease models through co-culture with hepatic cells (FIGS. 13a and 13b; FIGS. 14a and 14b; and Table 1).

TABLE 1

| | Drug | | | | | Effect on |
|---|---|---|---|---|---|---|
| | sensitivity/ | Suitability | | Collagen | PDGFR | liver |
| | reactivity | RGD | Fibrin | expression | expression | function |
| LX-2 | Poor | +++ | +++ | + | ++ | Inhibitory |
| K-HSC | Good | + | +++ | ++ | +++ | |

Also, in an embodiment of the present invention, the gene sequence or amino acid sequence information of the hepatocyte nuclear factor 4 alpha (HNF4u), albumin, vimentin, smooth muscle actin alpha (α-SMA), type I collagen (Col-1A1), lysyl oxidase (LOX), tissue inhibitor of matrix metalloproteinase 1 (TIMP1), platelet-derived growth factor beta (PDGFR-0), collagen type IV alpha 1 (Col-4A1), collagen type IV alpha 3 (Col-4A3), laminin subunit beta 1 (LamB1), fibronectin (FN), integrin subunit alpha 2 (ITGA2), integrin subunit beta 1 (ITGB1), cytochrome 3A4 (CYP3A4), CK-19, GFAP, desmin, and nestin may be obtained from a known database (e.g., GenBank of NCBI). In addition, the gene sequences or amino acid sequences of the hepatocyte nuclear factor 4 alpha (HNF4u), albumin, vimentin, smooth muscle actin alpha (α-SMA), type I collagen (Col-1A1), lysyl oxidase (LOX), tissue inhibitor of matrix metalloproteinase 1 (TIMP1), platelet-derived growth factor beta (PDGFR-0), collagen type IV alpha 1 (Col-4A1), collagen type IV alpha 3 (Col-4A3), laminin subunit beta 1 (LamB1), fibronectin (FN), integrin subunit alpha 2 (ITGA2), integrin subunit beta 1 (ITGB1), cytochrome 3A4 (CYP3A4), CK-19, GFAP, desmin, and nestin may have at least 70%, at least 75%, at least 80%, at least 85%, at least 90%, at least 95% or at least 99% sequence identity to the nucleotide sequence or amino acid sequence information which may be obtained from the known database.

Yet still another aspect of the present invention provides a method for preparing a liver fibrosis model including 3D co-culturing the transformed human hepatic stellate cell line and hepatic cells or a liver fibrosis model obtained by the method.

The transformed human hepatic stellate cell line and hepatic cells are the same as described above.

The 3D co-culture may be performed without a scaffold or with a scaffold, and the scaffold or scaffold-based culture is the same as described above. In addition, the 3D co-culture may be performed by adding a macromer to a culture medium in order to simulate the stiffness of liver fibrosis. The macromer may be at least 4%, at least 5%, at least 6%, at least 7%, or at least 8%, and 4% to 8% macromer may be used, but is not limited thereto. The liver fibrosis model may be a liver spheroid or an artificial liver construct, and the liver spheroid and the artificial liver construct are the same as described above. According to an embodiment of the present invention, it is confirmed that the liver fibrosis model exhibits a constant degree of hepatocellular function and a liver injury simulation regardless of the type of adhesion protein during the scaffold-based culturing.

According to an embodiment of the present invention, it is confirmed that the liver fibrosis model is capable of simulating the stiffness of liver fibrosis or liver fibrosis-associated diseases through the contraction of the culture during the scaffold-based culturing.

According to an embodiment of the present invention, the liver fibrosis or fatty liver model prepared using the transformed human hepatic stellate cell line has a higher sensitivity to TGF-β1, which is a liver fibrosis-inducing factor, than the liver fibrosis or fatty liver model prepared using a conventionally used human hepatic stellate cell line. In addition, since the transformed human hepatic stellate cell line does not have a direct effect on the differentiation or albumin production of hepatic cells even after long-term culture, the liver fibrosis or fatty liver model prepared using the transformed human hepatic stellate cell line is characterized by having improved sensitivity to fibrosis and metabolic degradation of hepatic cells caused by drugs or growth factors, and thus is possible to closely simulate the living environment and to secure a more sensitive response in disease modeling (see FIGS. 13a and 13b).

Another aspect of the present invention provides a method for evaluating a liver fibrosis regulator, the method including treating the liver fibrosis model including the liver spheroid or the artificial liver construct with a candidate material of regulating liver fibrosis.

The term “liver fibrosis regulation” herein may mean inhibiting or activating liver fibrosis, and the material that inhibits liver fibrosis may be a material that prevents or treats liver diseases such as liver fibrosis, liver disease associated with liver fibrosis, or fatty liver. The liver disease associated with liver fibrosis may include liver fibrosis, liver cirrhosis, cirrhosis, and liver cancer, but is not limited thereto.

Evaluating the liver fibrosis regulator may include evaluating the efficacy, safety, and toxicity of a candidate material that inhibits or activates liver fibrosis, or screening therapeutic agents of liver diseases such as liver fibrosis, liver disease associated with liver fibrosis, or fatty liver.

### Modes for the Invention

### EXAMPLES

Hereinafter, the present invention will be described in more detail with reference to Examples. These examples are for describing the present invention in more detail, and the scope of the present invention is not limited to these examples.

### Experimental Method 1. RT-PCR

For RT-PCR, the total RNA of the cell line was isolated using the TRIzol (Thermo Fisher; 15596018) reagent, and then synthesized into cDNA using TOPScript™ RT DryMIX (Ezynomics; RT200). RT-PCR was performed using synthesized cDNA as a template, the FAST SYBR® Green Master Mix (Applied Biosystems; 4385614) reagent and gene-specific primers. The used primers are shown in Table 2 below.

TABLE 2

| Target | | | | |
|---|---|---|---|---|
| gene | Primer (Forward) | SEQ ID NO. | Primer (Reverse) | SEQ ID NO. |
| HNF4α | GGC CAA GTA CAT CCC | SEQ ID NO: | CAG CAC CAG CTC GTC | SEQ ID NO: |
| | AGC TTT | 1 | AAG G | 2 |
| Albumin | TTTATGCCCCGGAACTCCTT | SEQ ID NO: | AGTCTCTGTTTGGCAGACGA | SEQ ID NO: |
| | T | 3 | A | 4 |
| Vimentin | GAGTCCACTGAGTACCGGAG | SEQ ID NO: | ACGAGCCATTTCCTCCTTCA | SEQ ID NO: |
| | | 5 | | 6 |
| α-SMA | GTGGCTATTCCTTCGTTACT | SEQ ID NO: | GGAAACGTTCATTTCCGATG | SEQ ID NO: |
| | | 7 | | 8 |
| Col-IA1 | ATTAGTAGGTGTGCTGTGTG | SEQ ID NO: | AAGCGTTTGCGTAGTAATTG | SEQ ID NO: |
| | | 9 | | 10 |
| LOX | GACACATCCTGTGACTATGG | SEQ ID NO: | GGGGTTTACACTGACCTTTA | SEQ ID NO: |
| | | 11 | | 12 |
| TIMP 1 | GACCACCTTATACCAGCGTT | SEQ ID NO: | TAAACAGGGAAACACTGTGC | SEQ ID NO: |
| | | 13 | | 14 |
| PDGFRβ | CCTCTTGGATATGCCTTACC | SEQ ID NO: | CAGCTCAGCAAATTGTAGTG | SEQ ID NO: |
| | | 15 | | 16 |
| Col-4A1 | ATTAGTAGGTGTGCTGTGTG | SEQ ID NO: | AAGCGTTTGCGTAGTAATTG | SEQ ID NO: |
| | | 17 | | 18 |
| LamB1 | GAGAGGTGATATTTCGTGCT | SEQ ID NO: | CAGAAGCAATTTCCTCGAAC | SEQ ID NO: |
| | | 19 | | 20 |
| FN | AGTTTCCCATTATGCCGTTG | SEQ ID NO: | TCCCAAGACATGTGCAGCTC | SEQ ID NO: |
| | | 21 | | 22 |
| ITGA2 | GGCCAGGTCATTATCTACAG | SEQ ID NO: | GGTTCCAGGCTCATTTGATA | SEQ ID NO: |
| | | 23 | | 24 |
| ITGB1 | AGAGCTGAAGACTATCCCAT | SEQ ID NO: | GTGTTGTGCTAATGTAAGGC | SEQ ID NO: |
| | | 25 | | 26 |
| CYP3A4 | TTTTGTCCTACCATAAGGGC | SEQ ID NO: | CATAAATCCCACTGGACCAA | SEQ ID NO: |
| | | 27 | | 28 |
| CK-19 | AGCATGAAAGCTGCCTTGGA | SEQ ID NO: | CCTGATTCTGCCGCTCACTA | SEQ ID NO: |
| | | 29 | TC | 30 |

### Experimental Method 2. Immunofluorescence Staining

For immunofluorescence staining, the cell line was fixed for 15 minutes using 4 paraformaldehyde 3-4 days after the culturing. Thereafter, the cell membrane of the cell line was permeabilized for 15 minutes using 0.25 Triton X-100. The primary antibodies were diluted in a buffer containing 400 bovine albumin and reacted at 4° C. for 16 hours, and then the secondary antibodies specific to the primary antibodies and attached with fluorescent materials were diluted and reacted at room temperature for 1 hour. An image corresponding to the marker was obtained using a fluorescence microscope. In this case, the primary antibodies used for immunofluorescence staining are as follows.

TABLE 3

| Antibodies | Catalog No. | Company | Dilution |
|---|---|---|---|
| anti-GFAP | Z0334 | DAKO | 1:200 |
| anti-desmin | AB907 | Millipore | 1:100 |
| anti-endoglin | AF1097 | R&D | 1:40 |
| anti-α-SMA | A5228 | Sigma | 1:100 |
| anti-nestin | MAB5326 | Millipore | 1:100 |
| Anti-type I collagen | ab34710 | abcam | 1:50 |
| anti-PDGFRB | ab32570 | abcam | 1:50 |

### I. Preparation of Immortalized Transformed Hepatic Stellate Cell Line

### Example 1. Preparation of Immortalized Transformed Hepatic Stellate Cell Line Kribb-HSC (K-HSC)

Human hepatic stellate cells were isolated from the liver tissues from patients (Chungnam National University Human Resources Bank) by treatment with 2 mg/mL collagenase I (Worthington, USA) at 37° C. for 30 minutes, and were recovered as single cells by filtration with a mesh of 100 μm pore size. The cells thus obtained were cultured for 16-18 hours in a primary hepatocyte maintenance media CM4000 (ThermoFisher) to which 10% FBS and 1% penicillin/streptomycin were added, and then pLenti-SV40 and pLenti-hTERT viruses (abm, US) were cultured together with 4 μg/mL polybrene (Sigma) for 16-18 hours, thereby being immortalized. Then, the cells were cultured in a hepatocyte maintenance medium.

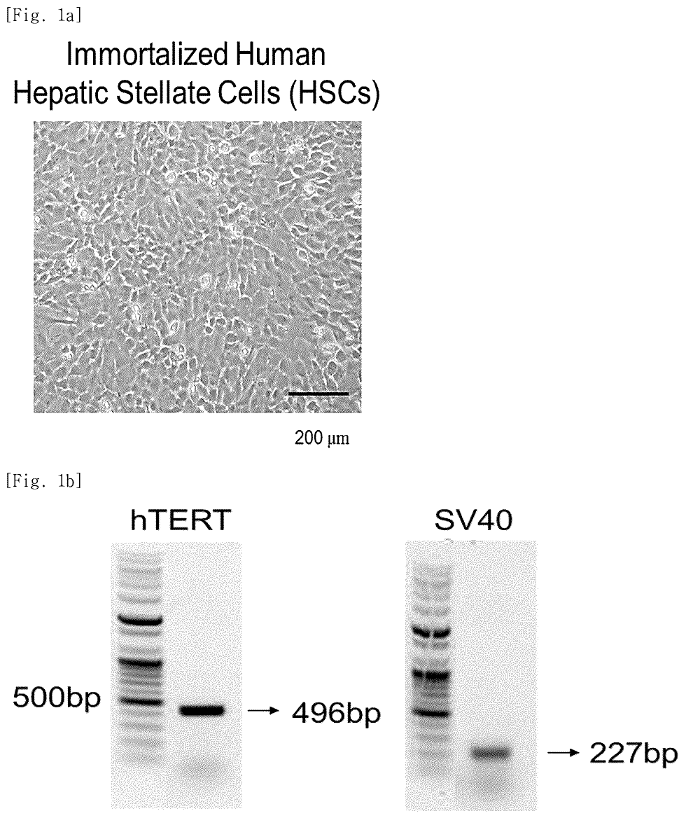

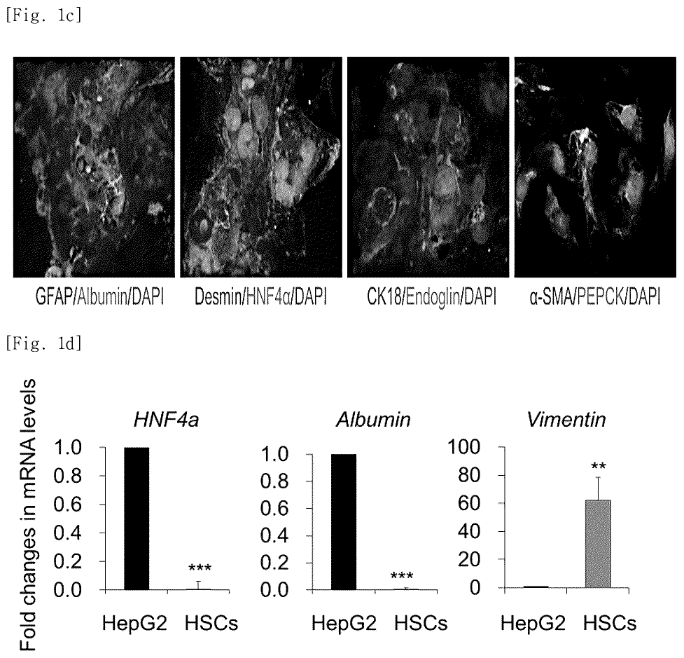

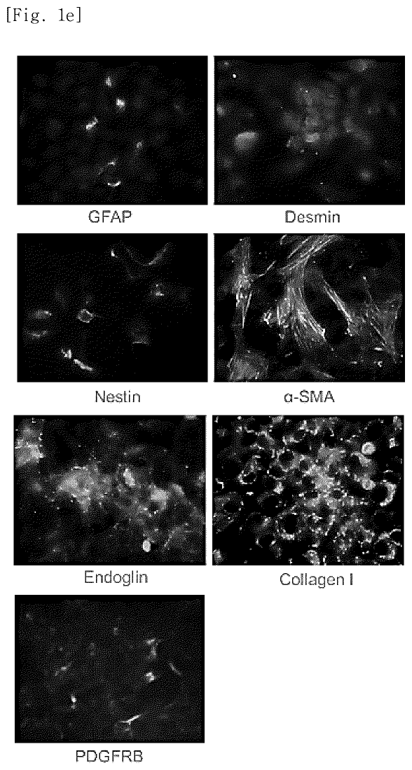

Single-cell colonies were cultured in hepatic stellate cell line medium (DMEM/F12 (Thermofisher 11330), 10% fetal bovine serum, and 1% penicillin/streptomycin) to secure immortalized human hepatic stellate cell lines (HSCs) that are capable of proliferating infinitely. The hepatic stellate cell lines were established from the single-cell colonies. Morphological characteristics of the cell line were confirmed through a microscope, and the expression of hTERT and SV40 large T-antigen was confirmed by PCR (FIGS. 1a and 1b).

In addition, the expression of the hepatic stellate cell markers (GFAP, desmin, endoglin, and α-SMA) of the prepared hepatic stellate cell line was confirmed by immunofluorescence staining, and the expression markers of the hepatic stellate cell line and the hepatic cell line HepG2 (ATCC) were confirmed by RT-PCR analysis.

As a result, the prepared hepatic stellate cell lines expressed weakly albumin and PEPCK, which are hepatocyte markers, hardly expressed HNF4u and CK18, and expressed GFAP, desmin, endoglin, and α-SMA, which are hepatic stellate cell line markers (FIG. 1c). Meanwhile, the HSCs showed remarkably low expression levels of HNF4u and albumin, which are hepatocyte markers, as compared with HepG2 hepatic cells, whereas the HSCs showed increased expression level of vimentin, which is a hepatic stellate cell marker (FIG. 1d).

To confirm whether the hepatic stellate cell lines maintain hepatic stellate cell characteristics even after subculturing, the subculturing was performed in a hepatic stellate cell line medium (DMEM/F12 (Thermofisher 11330), 10% fetal bovine serum, 1% penicillin/streptomycin) up to 19 times, and immunofluorescence staining was performed. As a result, it was confirmed that the cell line expressed GFAP, desmin, endoglin, nestin, α-SMA, type I collagen, and PDGFRB, which are hepatic stellate cell markers, even after subculturing (FIG. 1e).

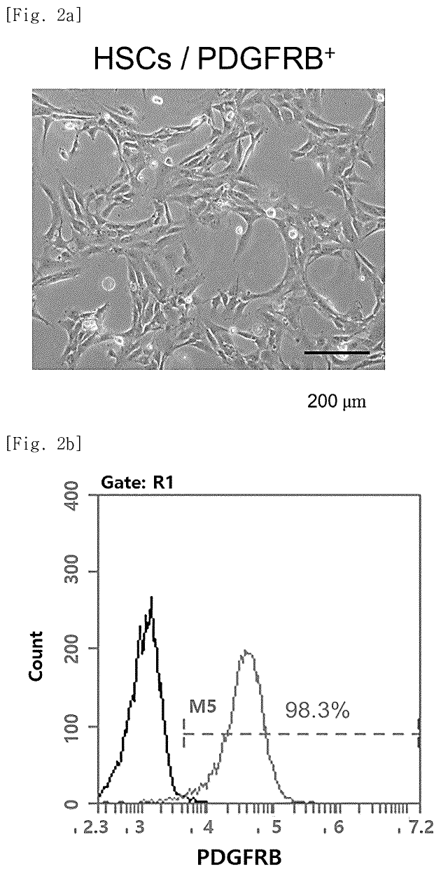

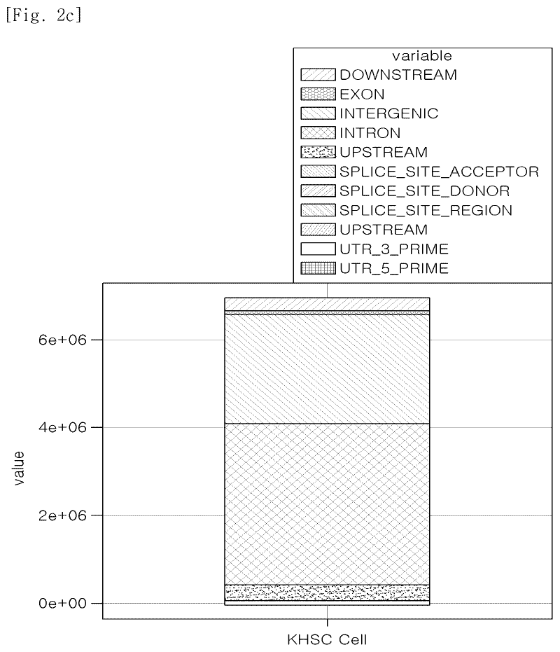

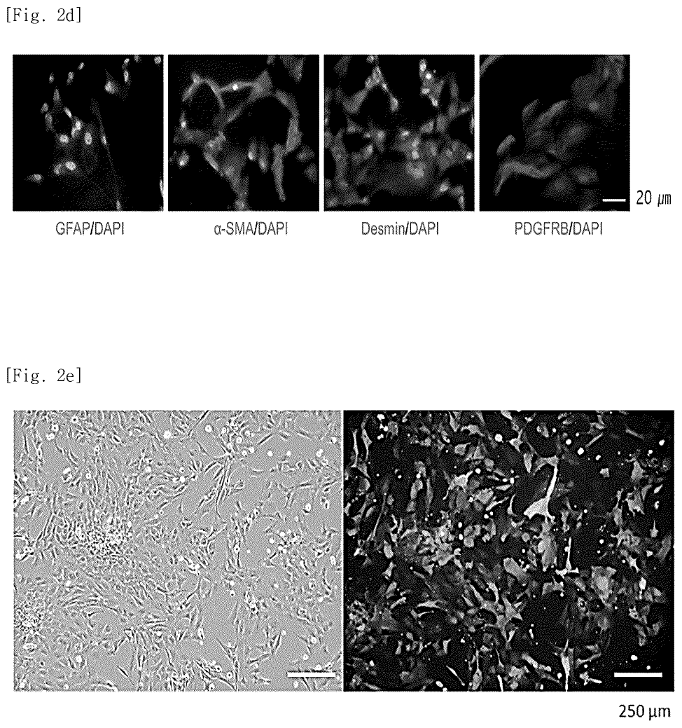

In order to isolate cells exhibiting the same characteristics of the hepatic stellate cells among the subcultured cells and to utilize the isolated cells as a disease model, PDGFRB⁺ (CD140b⁺) cells were isolated using a magnetic-activated cell sorting (MACS), and the isolated cells were identified using a fluorescence-activated cell sorting (FACS), and the purity of the PDGFRB⁺ cells was about 98.3% (FIGS. 2a and 2b). The cells were named KRIBB-HSC (K-HSC) cell lines, and insertion nucleotide sequences and variants were identified through full-length genome sequencing (FIG. 2c).

As a result of confirming, with immunofluorescence staining, the expression levels of GFAP, α-SMA, desmin, and PDGFRB, which are hepatic stellate cell markers, in the K-HSC cells, the hepatic stellate cell markers were well expressed (FIG. 2d).

### Example 2. Preparation of GFP-Hepatic Stellate Cell Line

For convenience of use in the future, K-HSC-GFP was prepared using retroviruses containing green fluorescence protein (GFP) genes. 1×10⁵ cells of the hepatic stellate cell lines prepared in Example 1 were seeded in a 6-well culture dish, and the next day, 1 hour before virus infection, the cells were pretreated with 8 ng/mL polybrene reagents (Santa Cruz Biotechnology, USA) in order to increase virus infection efficiency. Then, the cells were treated with GFP-retrovirus at multiplicity of infection (MOI) of 5 and cultured in a 5% CO₂ incubator at 37° C. for one day. The next day, after the medium was replaced with a fresh culture medium, the culture was continued, and after 48 hours, a strong GFP expression signal was confirmed to secure K-HSC-GFP hepatic stellate cell lines (FIG. 2e).

### Experimental Example 1. Determination of Fibrosis Activity of Hepatic Stellate Cell Line K-HSC

The hepatic stellate cell lines (K-HSC) prepared in Example 1 and the commercially available hepatic stellate cell line LX-2 (Millipore, USA) were stored in liquid nitrogen, and then the same lot was thawed and subcultured 5 times in Williams E medium (Sigma, USA) containing 2% FBS and DMEM medium (Sigma, USA), respectively. Thereafter, hepatic stellate cell-specific markers were compared through RT-PCR analysis. When the LX-2 was cultured in the DMEM medium, the expression of hepatic stellate cell-specific markers was set as 1, and relative mRNA expression was measured.

In the K-HSC according to the present invention, it was confirmed that α-SMA, Col-IA1, LOX, and TIMP1, which are liver fibrosis markers, and nestin and PDGFR-0, which are hepatic stellate cell markers, were more activated in Williams E medium, and in particular, the activity of α-SMA, which is a myofibroblast marker involved in tissue contraction, was increased by 10 times or more compared to the LX-2 (FIG. 3a). This was verified through cell fluorescence staining (FIG. 3b). From this, it may be seen that the K-HSC prepared in Example 1 has excellent fibrosis activity compared to the LX-2 cells.

The expression levels of markers (Col-4A3, Col-4A1, LamB1, FN, ITGA2, and ITGB1) for various materials constituting the extracellular matrix were confirmed by cell fluorescence staining and RT-PCR. As a result, the K-HSC of the present invention showed increased expression compared to the LX-2 (FIGS. 3b and 3c). This means that the adhesion between cell-cell and between cell-matrix is excellent and the cell health is excellent.

The expression levels of hepatocyte growth factor (HGF), and transforming growth factor beta 1 (TGF-β1), which is a fibrosis-inducing factor, were confirmed through RT-PCR. As a result, the K-HSC of the present invention and the existing LX-2 showed significantly lower expression than the undifferentiated hepatic cell line HepaRG, but in the case of TGF-β1, the LX-2 showed increased expression level compared to K-HSC (FIG. 3d). This means that the degree of fibrosis of the K-HSC of the present disclosure into myofibroblasts may be higher.

### II. Preparation of Liver Spheroid Through Co-Culture of Immortalized Transformed Hepatic Stellate Cell Lines and Hepatic Cell Lines

### Example 3. Three-Dimensional Co-Culture of Hepatic Stellate Cell Line K-HSC and Hepatic Cell Line (Self-Association)

In order to confirm the effect of the K-HSC hepatic stellate cell line prepared in Example 1 on hepatic cells, co-culture of hepatic stellate cell lines and hepatic cells was performed.

The commercially available hepatic cell line HepaRG (Biopredic, France) and the K-HSC prepared in Example 1 were applied at a ratio of hepatic cells/hepatic stellate cells of 4:6 and 8:2 onto the surface of a 48-well plate coated with 1.5% agarose using a Williams E medium-based serum-free medium containing hepatocyte growth factor (HGF, 10 ng/mL) and epidermal growth factor (EGF, 2 ng/mL), respectively, and 3D cultured while inducing self-association through the rotary shaking (70 rpm) without medium exchange for 7 days to form a spheroid. In this case, the total cell concentration was 3×10⁵ cells/mL. For comparison, only the hepatic cell line HepaRG was cultured in the same manner without hepatic stellate cell lines and set as a reference for the degree of hepatic cell differentiation. As a control group, the commercially available hepatic stellate cell line LX-2 was co-cultured with the hepatic cell line HepaRG in the same manner.

### Experimental Example 2. Confirmation of Hepatic Cell Activity and Fibrosis Activity of Liver Spheroid Prepared by 3D Co-Culture of Hepatic Stellate Cell Line K-HSC and Hepatic Cell Line

The effect of direct contact with the hepatic stellate cell line without a material-based matrix in the liver spheroid prepared in Example 3 on the differentiation degree of hepatic cells was confirmed by RT-PCR. As a result, it was confirmed that the K-HSC prepared in Example 1 did not inhibit the differentiation of hepatic cells because the K-HSC exhibited improved expression of HNF4α, albumin, and CYP3A4 enzyme, which are markers for the differentiation into hepatic cells (when the ratio of hepatic cells and hepatic stellate cell lines is 4:6) or exhibited similar expression levels thereof (when the ratio of hepatic cells and hepatic stellate cell lines is 8:2), compared to a spheroid composed of only hepatic cell line (HepaRG) when the K-HSC was co-cultured with the hepatic cell line (FIG. 4a). In addition, it was confirmed that the spheroid obtained by the co-culturing of the K-HSC according to the present invention and the hepatic cell line exhibited high expression in all of HNF4α, albumin and CYP3A4 enzyme, which are markers for the differentiation into hepatic cells, and CK-19, which is a marker for the differentiation into cholangiocytes, compared to the spheroid co-cultured with the LX-2, and thus did not show a decrease in the function of hepatic cells, which is better than the LX-2 (FIG. 4a).

Meanwhile, Col-1A1, α-SMA, TIMP1, and LOX, which are markers for the fibrosis progress of the hepatic stellate cells, exhibited the degree of initial liver fibrosis similar to that of LX-2 (FIG. 4b), unlike the 2D culture (FIGS. 3a and 3b) in which the markers in the K-HSC generally exhibited higher expression than those in the LX-2.

The above results show that the spheroid using the K-HSC prepared in Example 1 in a disease model such as liver fibrosis or fatty liver may simulate a biological environment more closely and secure more favorable initial conditions in disease modeling through the 3D culture. In addition, the results show that the K-HSC prepared in Example 1, unlike LX-2, improves the activity of the hepatic cell line and improves the sensitivity to fibrosis and metabolic degradation caused by drugs or growth factors.

### III. Preparation of Liver Analog Through Scaffold-Based Co-Culture of Transformed Hepatic Stellate Cell Line K-HSC and Hepatic Cell Line

The K-HSC prepared in Example 1 was encapsulated in a scaffold together with the hepatic cells, and then 3D co-culture was performed. In the same manner as in Example 3, HepaRG, which is an immortalized differentiated hepatic cell line, was used as the hepatocyte, and the LX-2, which is a commercially available hepatic stellate cell line, was used as a control group. After long-term culture in a 3D culture matrix, the network formation of cells was confirmed through a confocal microscope through actin-phalloidin fluorescence staining, and the activity of cells was confirmed through ATP analysis. In addition, drug metabolism, which is a major function of hepatic cells, was confirmed by measuring the activity of cytochrome p450 3A4 enzyme induced by rifampicin, and albumin production was confirmed through ELISA. In addition, the effect of direct contact between cell lines on the degree of differentiation of the hepatic cell line using a spheroid culture, which is a cell-based 3D culture model, was confirmed by RT-PCR. As a result, it was confirmed that the hepatic stellate cell line improves the activity of the hepatic cell line, unlike LX-2.

### Example 4. Preparation of Liver Analog Using Fibrin Gel Scaffold

When the hepatic stellate cell line K-HSC prepared in Example 1 and the LX-2 were subjected to immunofluorescence staining with PDGFR-beta, which is a hepatic stellate cell marker, and collagen, which is a marker for myofibroblasts differentiated from hepatic stellate cells, and was then compared, it was found that the markers for hepatic stellate cells and the differentiated myofibroblasts were all activated in the K-HSC unlike the LX-2 (FIG. 5a).

Meanwhile, when the hepatic stellate cell line K-HSC prepared in Example 1 was 3D cultured in a fibrin gel scaffold, whether the cell adhesion ability was improved and the network formation ability were confirmed.

Specifically, liver analogs were prepared by using, as a 3D culture matrix, a fibrin gel based on a natural material and a hybrid hydrogel containing a polyethylene glycol based on an artificial material/hyaluronic acid/RGD peptide as a control group.

The hepatic cell line HepaRG maintained in a Williams E medium containing 10% fetal bovine serum, antibiotics, insulin, hydrocortisone and the hepatic stellate cell line K-HSC prepared in Example 1 maintained in a medium of the same component except containing 2% fetal bovine serum were treated with trypsin. Thereafter, the HepaRG and the K-HSC were added to a solution of PBS and thrombin, and mixed with a solution of fibrinogen (GreenPlast Q®, Green Cross, Korea) of the same volume (diluted with PBS to ½, ⅙, and ⅛, respectively) to finally make 40 μL gel having a concentration of 2×10⁷ cells/mL and a ratio of hepatic cells/hepatic stellate cells of 5:5. Subsequently, the resulting gel was put into an incubator for 15 minutes to perform gelation.

Meanwhile, a hydrogel macromer based on polyethylene glycol (Sigma, USA) having 3 or 4 alkyl group spacers (Sigma, USA) was synthesized at a concentration of 6% in the trypsin-treated hepatic stellate cell line and hepatic cell line, and the gel was mixed with 0.36% of 1.5 MDa hyaluronic acid (Lifecore Biomedical, USA), and a crosslinkable 1 μM/mL RGD peptide (Bachem, USA, JenKem, China) to perform gelation using photopolymerization through ultraviolet irradiation. In this case, Irgacure® 2959 (Sigma, USA) as a photoinitiator was dissolved in a 70% ethanol solution, and then finally added thereto at a concentration of 0.1%, and the gelation was performed. Two slide cover glasses were fixed at a 1-mm interval and 30 μL of gel-cell-polymer solution was added in-between and the both sides were irradiated with ultraviolet rays for 4 minutes and 30 seconds, respectively, and then the hydrogel-based 3D liver analogs were polymerized. The gel thus obtained was transferred to a 24-well dish and cultured for 16 days (confocal microscope imaging, FIG. 5b) or 21 days (hepatocyte metabolism and activity measurement, FIGS. 5c to 5e) while replacing the medium with a medium containing 10% fetal bovine serum every two days. The cultures were washed with PBS and fixed with 4% paraformaldehyde. Next, actin was stained with phalloidin and the cell nucleus was stained with Hoechst to observe the formation of an intercellular network through a confocal microscope.

The K-HSC prepared in Example 1 showed excellent adhesion and network formation ability in the fibrin gel, but showed poor cell adhesion ability in the hybrid hydrogel compared to the LX-2 (FIG. 5b).

### Experimental Example 3. Confirmation of Hepatic Cell Activity of Liver Analogs Prepared Using Fibrin Gel Scaffold

In Example 4, the cells encapsulated in the fibrin gel were divided into two experimental groups while the medium was replaced the next day, and one group was cultured for 14 days in a hepatocyte differentiation medium containing 1.7% DMSO and the other group was cultured for 14 days in a normal medium containing 10% fetal bovine serum, and then in both groups, the media were replaced with normal media without DMSO, and the cells were cultured for three more days.

On the last culture day, the supernatant was collected, and albumin was quantified using the ELISA kit manufactured by Bethyl Laboratories, Inc., the medium was replaced with a medium containing 20 μM rifampicin, and the activity of the CYP3A4 enzyme was induced for 48 hours. The activity of the enzyme was confirmed by measuring the light intensity using a kit (P450-Glo CYP3A4 assay kit, V9002) manufactured by Promega Corporation.

Fibrin gel samples were dissolved using the CellTiter Glo kit manufactured by Promega Corporation, and then the ATP amount of the encapsulated cells was measured, and the affinity of the cells, the activity of the CYP3A4 enzyme, and the albumin production amount were normalized with the measured ATP amount and compared.

In addition, in the ATP analysis showing the cell activity, the hepatic stellate cell line K-HSC prepared in Example 1 did not show a difference from the LX-2, and thus, the fibrin gel affinity of the cells was similar (FIG. 5c). However, in the co-culture in which the ratio of the hepatic cell line HepaRG and the K-HSC or the LX-2 was 1:1, there was a difference in the activity of cytochrome 3A4 (CYP3A4) enzyme according to fibrin gel concentrations. It was confirmed that the amount of albumin secreted of HepaRG and the activity of CYP3A4 enzyme were higher in the co-culture of the K-HSC and HepaRG than in the co-culture control group of the LX-2 and HepaRG, and thus the K-HSC increased the activity of hepatic cells in the co-culture environment (FIGS. 5d and 5e).

### Example 5. Preparation of Liver Analogs Using Laminin-Coated Biosilk Foam Scaffold

The K-HSC prepared in Example 1 was subjected to 3D co-culture in another natural material-based laminin-coated biosilk foam. The hepatic cell line HepaRG and the hepatic stellate cell line were mixed at a ratio of 1:1 at a final concentration of 2×10⁷ cells/mL with a biosilk solution (Biosilk®521, BioLamina, Norway), then applied to wells of 24-well plate in a volume of about 40 μL, and gelated in a 37° C. cell incubator for 20 minutes, a medium was added thereto, and the medium was replaced every 2 days. As a result, the K-HSC exhibited excellent adhesion ability (FIG. 6a).

### Experimental Example 4. Confirmation of Hepatic Cell Activity of Liver Analogs Prepared Using Biosilk Scaffold

In Example 5, for a 3D co-cultured hepatic cell line in the laminin-coated biosilk foam, the cell affinity, CYP3A4 enzyme activity, and albumin secretion ability were measured in the same manner as in Experimental Example 2.

The hepatic cell line co-cultured in the laminin-coated biosilk foam was confirmed to have the affinity of the laminin-coated biosilk foam similar to the LX-2 like in the fibrin gel (FIG. 6b), and showed a difference in the activity of the CYP3A4 enzyme like the case using the fibrin gel scaffold in the co-culture with the hepatic cell line HepaRG, and showed high amount of albumin secreted and high activity of CYP3A4 enzyme compared to the co-culture control group with the LX-2 (FIGS. 6c and 6d). From this, it was confirmed that the K-HSC of the present invention increases the activity of hepatic cells in the co-culture environment with hepatic cells.

The above results showed that the hepatic stellate cell line of the present invention is a cell line applicable to the hepatocyte co-culture experiment.

### IV. Characterization of Hepatic Stellate Cell Line

### Experimental Example 5. Confirmation of Reactivity to Drug Administration of Hepatic Stellate Cell Line K-HSC According to Present Invention

To determine whether the K-HSC prepared in Example 1 is applicable as a drug-induced liver fibrosis model, 4%, 5% and 6% macromer concentrations were included to have various initial stiffness. Specifically, a hydrogel precursor based on polyethylene glycol (Sigma, USA) including 3 or 4 alkyl group spacers (Sigma, USA) and 1.5 MDa hyaluronic acid (Lifecore Biomedical, USA) were added at a ratio of 0.24% (4% macromer), 0.3% (5% macromer), and 0.36% (6% macromer), respectively, and finally mixed with a crosslinkable 1 μM/mL RGD peptide (Bachem, USA, JenKem, China), and then Irgacure®2959 (Sigma, USA) dissolved in a 70% ethanol solution was added at a concentration of 0.1% to perform polymerization. Two slide cover glasses were fixed at a 1-mm interval and 30 μL of gel-cell-polymer solution was added in-between and the both sides were irradiated with ultraviolet rays for 4 minutes and 30 seconds, respectively, and then the hydrogel-based 3D liver analogs were polymerized. The K-HSC and the hepatic cell line HepaRG were co-cultured at a ratio of 6:4. As a control group, the LX-2 was co-cultured in the same manner.

Specifically, hepatic stellate cells were encapsulated in the hybrid hydrogel at a concentration of 2×10⁷ cells/mL and a volume of about 30 μL. The hepatic cell line was cultured and differentiated in a DMSO environment for 14 days before the encapsulation, and the differentiated cells were collected and used. Primary human hepatic cells were collected from the liquid nitrogen in a frozen state and used immediately after thawing. After 24 hours of culture, the medium was converted to a serum-free medium. Thereafter, the culture was performed for 3 days, and a cycle of 24-hour drug administration-24-hour serum-free medium was performed 7 times. Experiments were performed in four groups: a control group (cont) to which a drug was not administered; experimental groups to which acetaminophen (apap) and methotrexate (MTX) were administered, respectively; and a positive control group to which TGF-β1 inducing fibrosis was administered. Thereafter, the cells were collected and the expression levels of α-SMA, Col-IA1, LOX, and TIMP-1, which are fibrosis markers, were measured by RT-PCR to confirm the degree of fibrosis progress of the hepatic stellate cells. As a result, referring to FIGS. 7a to 7d, it was found that the K-HSC prepared in Example 1 had higher drug sensitivity and better induced liver fibrosis than the LX-2, particularly in the 5% macromer matrix.

In addition, the expression levels of HNF4α and albumin, which are hepatocyte markers, and the expression level of fibronectin, which is a cell adhesion protein, were quantitatively analyzed by RT-PCR. As a result, the K-HSC prepared in Example 1 was confirmed to be sensitive to drug reaction due to a large difference between the expression of HNF4α indicating the degree of differentiation of hepatic cells and the expression of albumin indicating the function of hepatic cells according to the drug administration, and was confirmed not to have a direct effect on the differentiation of hepatic cells or the albumin production ability (FIGS. 8a and 8b). In addition, the K-HSC of the present invention was also confirmed to exhibit an excellent expression level of fibronectin, which is a fibrosis index (FIG. 8c). On the other hand, in the control group in which the LX-2 was used, overall low differentiation and function were shown so that there was no difference according to the drug administration. Meanwhile, the K-HSC prepared in Example 1 was encapsulated alone in the hydrogel, and 3D cultured alone without drug treatment, and the shape of the hydrogel was then observed. Cell-hydrogel encapsulation was performed using photopolymerization through ultraviolet irradiation. In this case, Irgacure® 2959 (Sigma, USA) as a photoinitiator was dissolved in a 70% ethanol solution, and then finally added thereto at a concentration of 0.1%, and the gelation was performed. The fixing was performed with hydrogel at a final cell concentration of 2×10⁷ cells/mL. Two slide cover glasses were fixed at a 1-mm interval, a gel macromer-cell-polymer solution containing 30 μL of RGD was added in-between, and the both sides were irradiated with ultraviolet rays for 4 minutes and 30 seconds, respectively, and then a culture containing 3D hepatic stellate cells was polymerized.

It was confirmed that the hepatic stellate cell line prepared in Example 1 exhibited a larger cell condensation than the LX-2 cell group, thereby having excellent fibrosis activation (FIG. 8d). Therefore, it was confirmed that the hepatic stellate cell line prepared in Example 1 may become a cell line of a liver fibrosis model superior to the LX-2.

### Experimental Example 6. Confirmation of Initial Reactivity According to Stiffness of Support of Hepatic Stellate Cell Line K-HSC According to Present Invention and Confirmation of Reactivity to Hepatocyte Growth Factor Removal

The K-HSC prepared in Example 1 and the hepatic cell line HepaRG were encapsulated at a ratio of 1:1 in a hybrid hydrogel system simulating the stiffness of normal liver tissue (4.5% macromer) or fibrosis disease liver tissue (8% macromer), and changes in initial fibrosis markers according to whether the hepatic stellate cell line reacts to the change in the stiffness of the support and whether the hepatocyte growth factor (HGF) is administered were confirmed. The LX-2 was used as a control group. The hepatic cell line HepaRG and the hepatic stellate cell line or the LX-2 were mixed at a ratio of 1:1 in hydrogels with different concentrations of each macromer, were fixed with hydrogels at a total cell concentration of 2×10⁷ cells/mL, and was mixed with 1 mM/mL RGD and 0.1% Irgacure® 2959 initiator, followed by photopolymerization for 9 minutes to prepare 3D liver analogs. The 3D liver analogs were cultured in media containing 10% bovine serum for one day, then the media were replaced with a serum-free medium to which 10 ng/mL of HGF was added (control medium) and a medium to which the HGF was not added, and then the replacement was performed once every two days, and the 3D liver analogs were cultured for 8 more days (total 9 days: initial conditions for constructing liver mimics). The 3D liver analogs were recovered and then treated with Trizol (ThermoFisher, USA), followed by RT-PCR analysis.

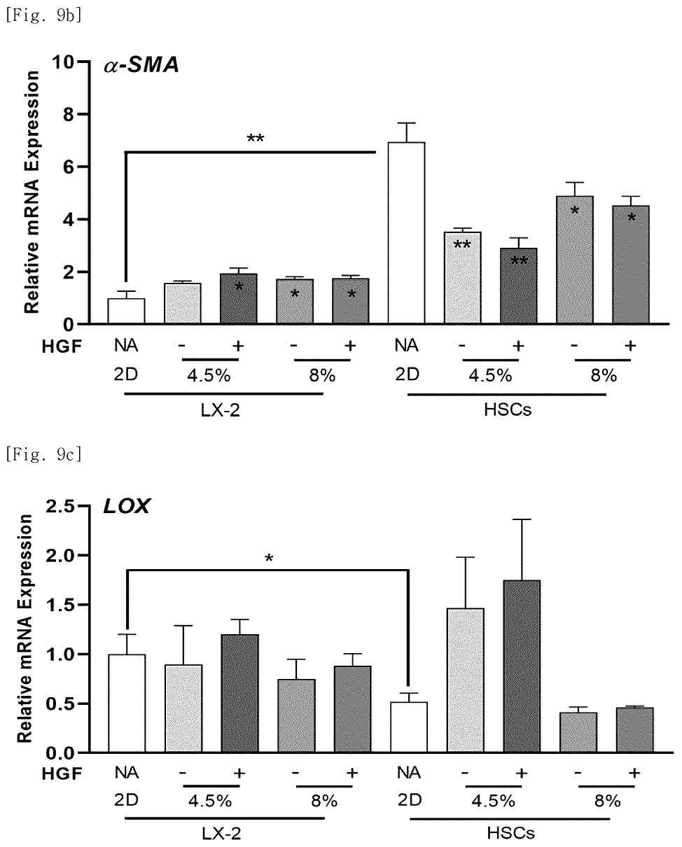

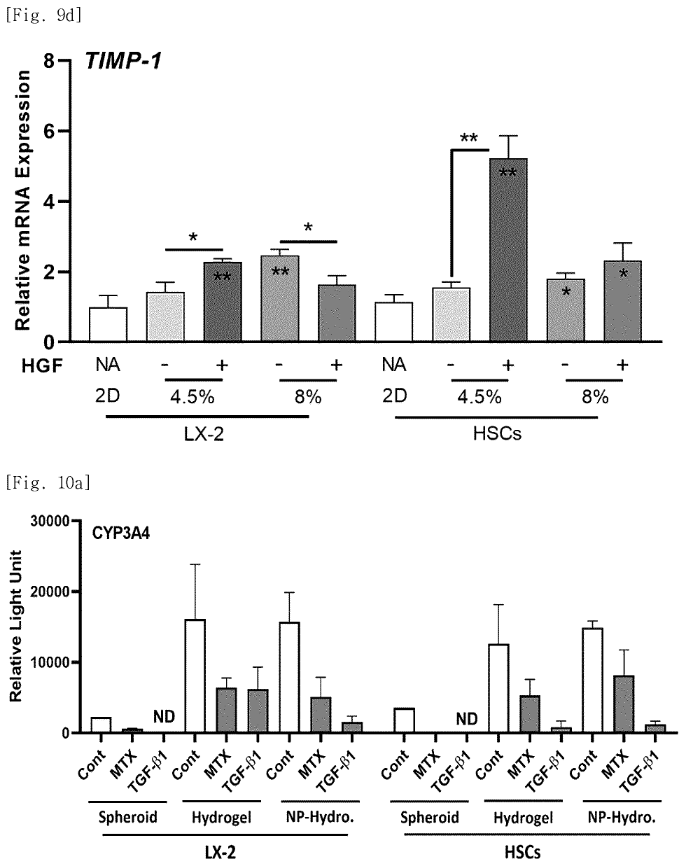

Referring to FIGS. 9a to 9d, it may be seen that the expression of Col-1A1, α-SMA, LOX, and TIMP1, which are fibrosis markers, was statistically significantly changed compared to the LX-2 control group according to the difference in the initial stiffness of the support (4.5%:8%), and in particular, the expression levels of Col-1A1 and α-SMA increased according to the change in stiffness, which means that the K-HSC cell line shows reactivity to stiffness. According to the presence or absence of the HGF growth factor, only the TIMP-1 gene showed a significant change, and it was confirmed that the expression increased when the stiffness was low in the medium to which HGF was added, and decreased similarly to the value at high stiffness when the HGF was removed. That is, it was confirmed that, in the 4.5% macromer hydrogel having low stiffness, the marker expression may be regulated according to whether the HGF is added.

### Experimental Example 7. Confirmation of Liver Injury Simulation According to Culture of Immortalized Transformed Hepatic Stellate Cell Line K-HSC According to Present Invention (Spheroid, Nanoparticle-Hydrogel, and Hydrogel Using Self-Association)

In order to compare liver injury simulation according to the 3D culture of the K-HSC prepared in Example 1, for one 3D culture, the K-HSC and hepatic cell line HepaRG were encapsulated and cultured in a hybrid hydrogel having a concentration of 5% macromer, and for the other, the K-HSC and hepatic cell line HepaRG were cultured in the form of a spheroid using self-association, and as a comparative group, the K-HSC and hepatic cell line HepaRG were cultured in a biodegradable polylactide nanoparticle-hydrogel (NP-hydrogel). As a control group, the LX-2 was co-cultured in the same manner as described above.

Thereafter, methotrexate (MTX) was added for the purpose of simulating liver injury, or TGF-β1 was added for the purpose of inducing liver fibrosis, and then the activity and average expression amount (activity rate of enzyme) of the CYP3A4 enzyme, and albumin production ability were confirmed in the same manner as in Experimental Example 2.

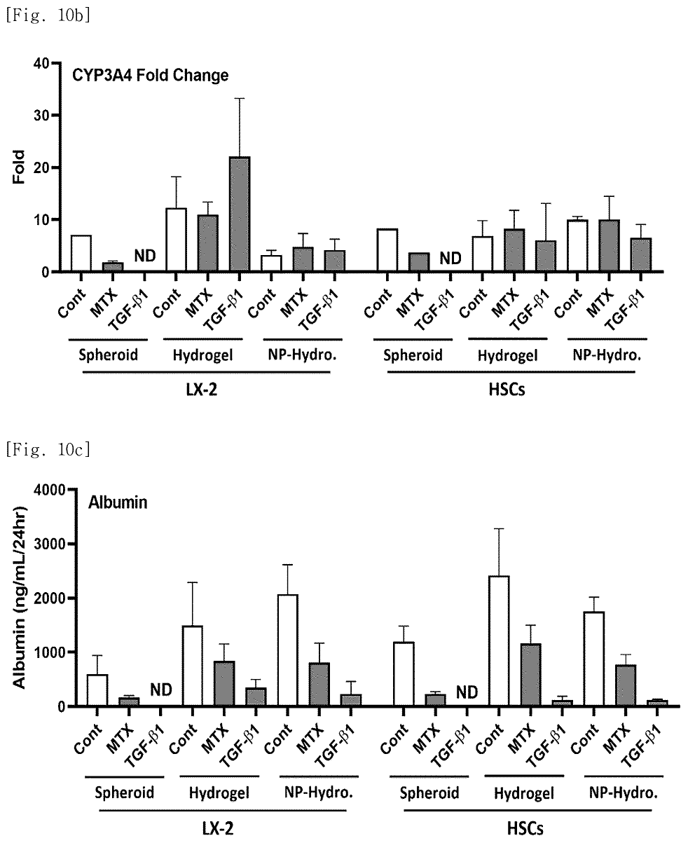

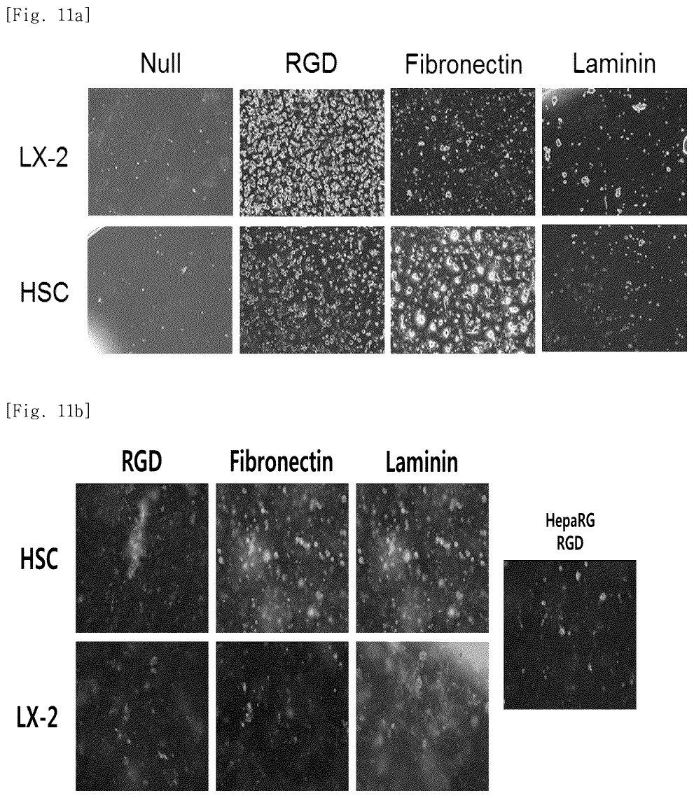

As a result, it was confirmed that both the K-HSC and the LX-2 exhibited low activity of the CYP3A4 enzyme and low albumin production ability when cultured in the form of a spheroid using self-association, and simulated a deterioration in the activity and function according to drug and induction effects when cultured in a hydrogel or a nanoparticle-hydrogel (FIGS. 10a to 10c). From this, it was confirmed that the nanoparticle hydrogel or hydrogel system was more suitable for liver injury simulation than the spheroid system using self-association.

### Experimental Example 8. Confirmation of Affinity, Hepatocellular Function, and Liver Injury Simulation According to Adhesion Protein of Hepatic Stellate Cell Line K-HSC According to Present Invention

In order to compare the affinity with various adhesion proteins in a 3D hydrogel environment, the hydrogel containing the RGD peptide and the LX-2 cell line were used as a control group, the surface adhesion was observed through an optical microscope after the 12-hour 2D-culture in a 5% macromer hybrid hydrogel in which fibronectin and laminin were encapsulated together, and the K-HSC and the LX-2 each were 3D cultured with the hepatic cell line HepaRG for 5 days in a hydrogel in which an adhesion protein was encapsulated, and then observed through a confocal microscope, thereby comparing the network formation of cells.

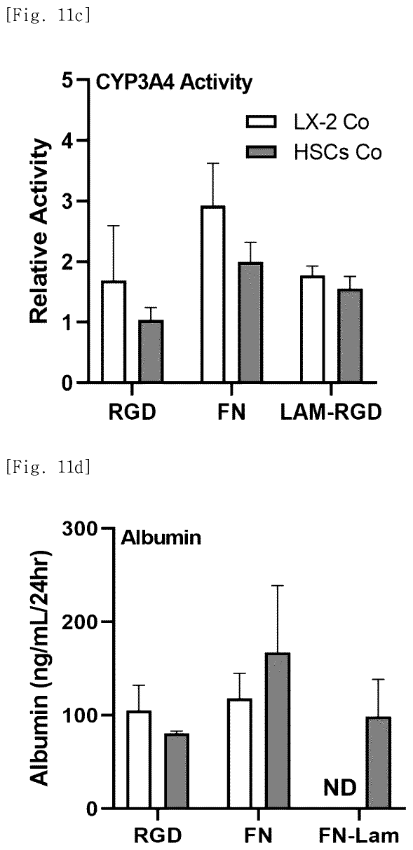

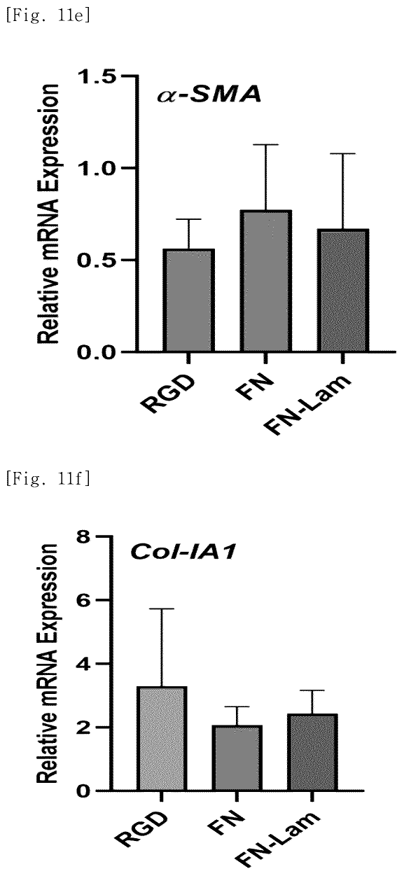

Referring to FIGS. 11a and 11b, it was confirmed that there were few differences in the adhesion pattern or density according to the adhesion proteins in the 2D and 3D cultures.

Meanwhile, in order to determine the effect on the function of the hepatocyte according to the adhesion proteins, the hepatic stellate cell line (LX-2 or K-HSC) and the differentiated HepaRG were encapsulated at a ratio of 1:1 in a hydrogel and cultured, thereby comparing the formation of the CYP3A4 enzyme and albumin according to the adhesion proteins. In order to determine the effect on the liver fibrosis simulation according to the adhesion proteins, the expression levels of smooth muscle actin alpha (α-SMA) and type I collagen (Col-1A1) were compared, but all of the liver fibrosis simulation, the hepatocellular function, and the liver injury simulation did not show statistically significant differences even though the adhesion protein types were different (FIGS. 1c to 11f).

This shows that the hepatocellular function and liver injury simulation properties of the K-HSC according to the present invention are constant regardless of the type of adhesion protein.

### Experimental Example 9. Confirmation of Liver Injury Simulation According to Culture of Hepatic Stellate Cell Line K-HSC According to Present Invention (Nanoparticle-Hydrogel and Hydrogel)

The K-HSC prepared in Example 1 was encapsulated with primary human hepatic cells (PHHs, Corning, USA) at a ratio of 1:1 in 4.5% polyethylene glycol/hyaluronic acid/RGD hydrogel using photopolymerization as described in Example 4. The cells frozen with nitrogen were thawed using recovery media (ThermoFisher, USA) and centrifuged, and then the number of cells were measured, and the cells were encapsulated with the K-HSC in a hydrogel, and then cultured for one day in a primary human hepatocyte plating medium (ThermoFisher, USA) containing 10% bovine serum. The medium was converted to a dedicated serum-free medium for primary human hepatic cells (Corning, USA), after 5 days, TGF-β1 inducing fibrosis, was added for 24 hours at a concentration of 10 ng/mL, and then converted back to a serum-free medium to which the TGF-β1 was not added in the following day, 3 cycles were performed, and the appearance change of the hydrogel was observed after the 24-day culture, and albumin production ability and expression levels of fibrosis markers were measured by RT-PCR.

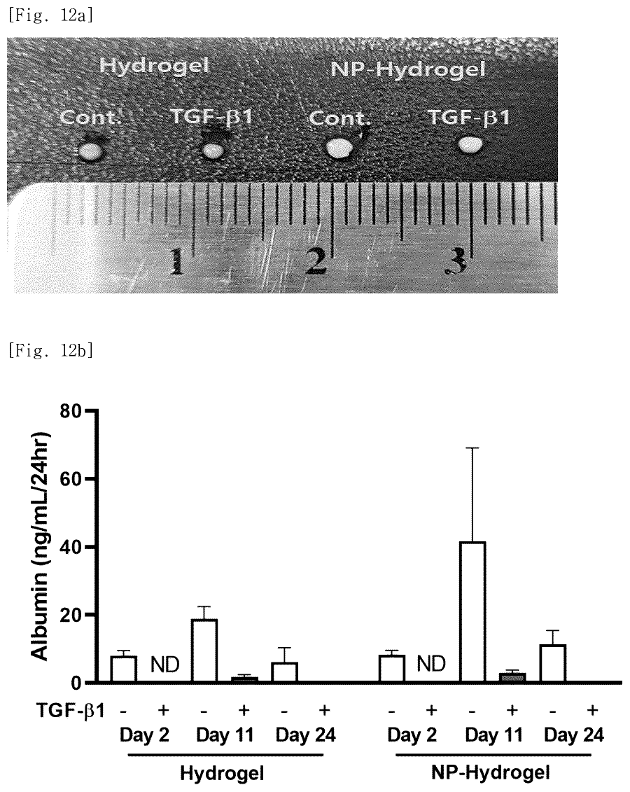

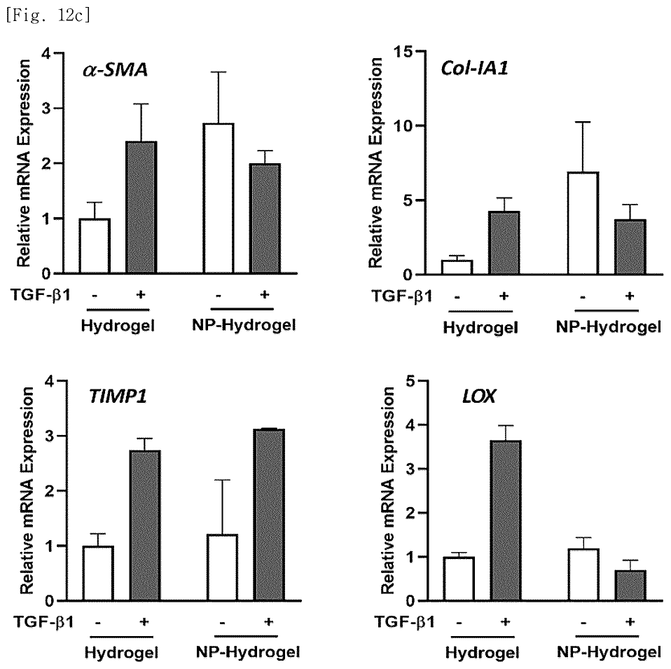

As a result, the nanoparticle-hydrogel and the hydrogel in which the K-HSC and the primary human hepatic cells were co-cultured did not show any appearance changes in the control groups including or not including TGF-β1 (FIG. 12a), and showed a deterioration in albumin degrading ability in the presence of TGF-β1 compared to the control groups (FIG. 12b). Meanwhile, in the expression of a liver fibrosis marker, nanoparticle-hydrogel did not show a significant change compared to the control group (FIG. 12c). From this, studies/clinical trials involving primary human hepatic cells have confirmed that the hydrogel system was more suitable for liver injury simulation.

### Experimental Example 10. Confirmation of TGF-β1 Sensitivity of Hepatic Stellate Cell Line K-HSC According to Present Invention

The K-HSC prepared in Example 1 and the differentiated hepatic cell line HepaRG were applied at a ratio of hepatic cells/hepatic stellate cells of 6:4 onto the surface of a 48-well plate coated with 1.5% agarose using a serum-free medium based on Williams E medium, and were self-associated in the presence of physical stimulus through the rotary shaking (70 rpm) for a total of 7 days in Williams E medium containing 2% serum for 3 days to form spheroids in the form of 3D culture. In this case, the total cell concentration was 3×10⁵ cells/spheroid. Thereafter, the medium was replaced with a serum-free Williams E medium on day 3 of culture, culture was performed for 4 days, and then TGF-β1, which is a liver fibrosis-inducing factor, was added thereto for 24 hours, and then the cells were exposed again to the medium without growth factor. This was set as one cycle, and the cycle was repeated 5 times (5 cycles), and then the cells were collected, and quantitatively analyzed with RT-PCR. The differentiated hepatic cell line HepaRG was cultured and differentiated in a long-term DMSO environment for 14 days or more before the formation of a 3D spheroid, and the differentiated cells were collected and used, and as a control group, the LX-2 was co-cultured with the differentiated hepatic cell line HepaRG in the same manner.

As a result, the co-culture spheroid composed of the K-HSC and the hepatic cell line HepaRG showed an increased expression level difference according to TGF-β1 introduction in the expression of smooth muscle actin alpha (α-SMA), type I collagen (Col-1A1), TIMP-1, TIMP-2, and fibronectin, which are liver fibrosis markers, compared to the co-culture spheroid composed of the LX-2 and the hepatic cell line HepaRG (FIG. 13a). This means that the K-HSC of the present invention has an overall TGF-β1 sensitivity higher than the LX-2.

In addition, it was confirmed that the expression of HNF4α, albumin, CYP2B6, and CYP3A4 enzyme, which are markers for the differentiation into hepatic cells, was maintained in a relatively high state in the control groups to which TGF- β1 was not added, and it was confirmed that CK19, which is a marker for the differentiation into cholangiocytes, also showed a high expression level (FIG. 13b). This means that the K-HSC of the present invention does not have a direct effect on the differentiation of hepatic cells or production of albumin.

Thus, it was confirmed that the K-HSC may simulate the biological environment more closely, and secure more sensitive reactivity in disease modeling through 3D culture.

Meanwhile, the K-HSC was encapsulated with the hepatic cell line HepaRG at a ratio of 1:1 in a 4% hydrogel and then 3D cultured, and the groups were divided into experimental groups to which TGF-β1, which is a fibrosis-inducing material, and inhibitor A thereof were added individually or simultaneously, and a control group to which TGF-β1 and inhibitor A were not added. The groups were cultured for 18 days, and then the cell network formation and expression differences of fibrosis markers in the liver analogs were confirmed.

As a result, there was a significant difference in the formation of the network in the hydrogel after 8 days of culture (FIG. 14a), and in the expression of the liver fibrosis markers, reactivity to TGF-β1 was confirmed in all markers except smooth muscle actin alpha (α-SMA) and lysyl oxidase (LOX), and TGF-β1 inhibitory action of inhibitor A was also confirmed (FIG. 14b).

From the above description, those of ordinary skill in the art of the present invention will be understood that the present invention can be carried out in other specific forms without changing the technical idea or essential features. Therefore, the above-disclosed embodiments are to be understood in all aspects as illustrative and not restrictive. Accordingly, the scope of the present invention is defined by the following claims rather than by the detailed description. It shall be understood that all modifications or changes in forms conceived from the meaning and scope of the claims and their equivalents are included in the scope of the present invention.

1. A transformed human hepatic stellate cell line into which a human telomerase reverse transcriptase (hTERT) gene and a SV40 large T-antigen gene are introduced and which shows positive for expression of a platelet-derived growth factor receptor beta (PDGFR-β) marker.

2. The transformed human hepatic stellate cell line of claim 1, wherein the hepatic stellate cell line has overexpressed smooth muscle actin alpha (α-SMA).

3. The transformed human hepatic stellate cell line of claim 1, wherein the hepatic stellate cell line exhibits increased expression of a hepatic stellate cell marker.

4. The transformed human hepatic stellate cell line of claim 3, wherein the hepatic stellate cell marker is at least one selected from the group consisting of smooth muscle actin alpha (α-SMA), lysyl oxidase (LOX), tissue inhibitor of matrix metalloproteinase 1 (TIMP1), and platelet-derived growth factor receptor beta (PDGFR-β).

5. The transformed human hepatic stellate cell line of claim 3, wherein the level of the increased expression of the hepatic stellate cell marker is at least 1.5 when the expression level of a hepatic stellate cell marker of an LX-2 cell line is set as 1.

6. The transformed human hepatic stellate cell line of claim 3, wherein the hepatic stellate cell line exhibits increased expression of one or more extracellular matrix markers selected from among collagen type IV alpha 1 (Col-4A1), collagen type IV alpha 3 (Col-4A3), laminin subunit beta 1 (LamB1), fibronectin (FN), integrin subunit alpha 2 (ITGA2), and integrin subunit beta 1 (ITGB1).

7. The transformed human hepatic stellate cell line of claim 1, wherein the hepatic stellate cell line exhibits liver fibrosis activity.

8. A cell composition for producing an artificial liver construct, the cell composition comprising the transformed human hepatic stellate cell line according to claim 1 and hepatic cells.

9. The cell composition of claim 8, wherein the hepatic cells are primary human hepatic cells, immortalized hepatic cell lines, or stem cell-derived hepatic cells.

10. A liver spheroid prepared by culturing the cell composition according to claim 8.

11. An artificial liver construct produced by culturing the cell composition according to claim 8 with a scaffold.

12. The artificial liver construct of claim 11, wherein the scaffold is a polyethylene glycol-containing hydrogel, a fibrin gel, or biosilk.

13. A method for preparing a liver fibrosis model, the method comprising 3D co-culturing the transformed human hepatic stellate cell line according to claim 1 and hepatic cells.

14. The method of claim 13, wherein the hepatic cells are primary human hepatic cells, immortalized hepatic cell lines, or stem cell-derived hepatic cells.

15. A liver fibrosis model obtained by the method according to claim 13.

16. A method for evaluating a liver fibrosis regulator, the method comprising treating the liver spheroid according to claim 10 with a candidate material of regulating liver fibrosis.

17. A method for evaluating a liver fibrosis regulator, the method comprising treating the artificial liver construct according to claim 11 with a candidate material of regulating liver fibrosis.
